SEQUENCE LISTING (8 sequences)

SEQ ID NO: 1 - protein, 10 aa, Artificial Sequence
  Synthetic peptide epitope
ELAGIGILTV

SEQ ID NO: 2 - protein, 66 aa, Artificial Sequence
  Synthetic peptide epitope
GILTVILGVLLLIGCWYCRRRNGYRALMDKSLHVGTQCALTRRCPQEGFDHRDSKVSLQEKNCEPV

SEQ ID NO: 3 - protein, 9 aa, Artificial Sequence
  Artificial peptide epitope
MLLAVLYCL

SEQ ID NO: 4 - protein, 9 aa, Artificial Sequence
  Artificial peptide epitope
YMDGTMSQV

SEQ ID NO: 5 - DNA, 3635 bp, Artificial Sequence
  Plasmid vector pSEM-U6-GFP
atatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagccaccatgttactagctgttttgtactgcctggaactagcagggatcggcatattgacagtgtatatggatggaacaatgtcccaggtaggaattctgacagtgatcctgggagtcttactgctcatcggctgttggtattgtagaagacgaaatggatacagagccttgatggataaaagtcttcatgttggcactcaatgtgccttaacaagaagatgcccacaagaagggtttgatcatcgggacagcaaagtgtctcttcaagagaaaaactgtgaacctgtgtagtgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgacccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgggatccggttatgtacaggaacgcattcaagagatgcgttcctgtacataacctttttggaaaagcttggcactggccgtcgtttttccggaagagtcaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtccggccggaaacgtttggttgctgactaattgagatgcatgctttgcatacttctgcctgctggggagcctggggactttccacacctcgcgatgtacgggccag

SEQ ID NO: 6 - DNA, 3636 bp, Artificial Sequence
  Plasmid vector pSEM-U6-Melan-A
atatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagccaccatgttactagctgttttgtactgcctggaactagcagggatcggcatattgacagtgtatatggatggaacaatgtcccaggtaggaattctgacagtgatcctgggagtcttactgctcatcggctgttggtattgtagaagacgaaatggatacagagccttgatggataaaagtcttcatgttggcactcaatgtgccttaacaagaagatgcccacaagaagggtttgatcatcgggacagcaaagtgtctcttcaagagaaaaactgtgaacctgtgtagtgagcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgacccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttgggtttatatatcttgtggaaaggacgcgggatccatcggctgttggtattgtattcaagagatacaataccaacagccgatttttttggaaaagcttggcactggccgtcgtttttccggaagagtcaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtccggccggaaacgtttggttgctgactaattgagatgcatgctttgcatacttctgcctgctggggagcctggggactttccacacctcgcgatgtacgggccag

SEQ ID NO: 7 - DNA, 3596 bp, Artificial Sequence
  Plasmid vector pBPL
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagccaccatgtccctgttgatgtggatcacgcagtgcaaagcttcggagaaaatcttctatgtgggtcttccaagtattcctgttcatccaattggtcttccaagtattcctgttcatccaattaaagcttcggagaaaatcttctatgtgtccctgttgatgtggatcacgcagtgcaaagcttcggagaaaatcttctatgtgaaagcttcggagaaaatcttctacgtacggtgcggtgccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctagtgagaattctgcagatatccatcacactggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctccggaagagtcaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtccggccggaaacgtttggttgctgactaattgagatgcatgctttgcatacttctgcctgctggggagcctggggactttccacacctcgcgatgtacgggccagatatacgc

SEQ ID NO: 8 - DNA, 3884 bp, Artificial Sequence
  Plasmid vector pROC
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagccaccatgaatctccttcacgaaaccgactcggctgtggccaccgcgcgccgcccgcgctggctgtgcgctggggcgctggtgctggcgggtggcttctttctcctcggcttcctcttcgggtggtttataaaaagcgctcagctggcaggggccaaaggagtcattctctactccgaccctgctgactactttgctcctggggtgaagtcctatccagatggttggaatcttcctggaggtggtgtccagcgtggaaatatcctaaatctgaatggtgcaggagaccctctcacaccaggttacccagcaaatgaatatgcttataggcgtggaattgcagaggctgttggtcttccaagtattcctgttcatcctattgccctgcagagtctcttgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaacaagcgatcgctcctgcaacacctcatcgggctgggggacgccgcctacagtctcctgcaacacctcatcgggctgatttccccggagaaggaagagcagtatatcgccagtctcctgcaacacctcatcgggctgaagaggccaagtattaagaggggtcttccaagtattcctgttcatccagtttagtgagaattctgcagatatccatcacactggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctccggaagagtcaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtccggccggaaacgtttggttgctgactaattgagatgcatgctttgcatacttctgcctgctggggagcctggggactttccacacctcgcgatgtacgggccagatatacgc

(19) United States
(12) Patent Application Publication
(10) Pub. No.: US 2011/0230548 A1
(43) Pub. Date: Sep. 22, 2011

(54) MULTICISTRONIC VECTORS AND METHODS FOR THEIR DESIGN

(75) Inventors: Adrian Ion Bot, Valencia, CA (US); David C. Diamond, West Hills, CA (US); Zhiyong Qiu, San Gabriel, CA (US)

(73) Assignee: MANNKIND CORPORATION, Valencia, CA (US)

(21) Appl. No.: 13/032,635

(22) Filed: Feb. 22, 2011

Related U.S. Application Data
(63) Continuation of application No. 12/126,855, filed on May 23, 2008, now abandoned.
(60) Provisional application No. 60/939,837, filed on May 23, 2007.

(51) Int. Cl. A61K 48/00; C12N 15/85; C12N 5/10; C12N 15/86

(52) U.S. Cl. 514/44 R; 435/320.1; 435/325

(57) ABSTRACT

Embodiments of the present invention relate to multicistronic vectors and methods for their design. Methods and compositions of the invention include a vector including at least two cistrons, wherein a first cistron includes a first promoter and a first nucleic acid sequence encoding one or more therapeutic agents, and wherein a second cistron comprises a second promoter and a second nucleic acid sequence encoding one or more RNA molecules that interfere with the expression of a biological response modifier or the therapeutic agent, wherein the expression of the first sequence is under control of the first promoter and expression of the second sequence is under control of the second promoter.

### CROSS-REFERENCE TO RELATED APPLICATIONS

This application claims priority from U.S. Provisional Application Ser. No. 60/939,837, filed on May 23, 2007, which is incorporated herein by reference in its entirety.

### INCORPORATION-BY-REFERENCE OF MATERIAL SUBMITTED ON A COMPACT DISC

The present application is being filed along with a Sequence Listing in electronic format. The Sequence Listing is provided as a file entitled 002_080523_SeqListing_MANNK_{—}058A.TXT, created May 23, 2008, which is 20 Kb in size. The information in the electronic format of the Sequence Listing is incorporated herein by reference in its entirety.

### FIELD OF THE INVENTION

The invention disclosed herein generally relates to multicistronic vectors and methods for their design and construction for use as immunotherapeutics capable of inducing an immune response in a subject or capable of suppressing a gene or target expressing an antigen.

### BACKGROUND

DNA based immunization refers to the induction of an immune response to a protein antigen expressed in vivo following the introduction of plasmid DNA into the host cell. In many instances, the design of DNA vaccines is relatively simple. Although these vaccines have been promising in mice, their efficacy in humans remains at issue as higher doses of the vaccine can be required in order to elicit a detectable immune response in humans compared to those required in mice.

### SUMMARY OF THE INVENTION

Embodiments of the present invention relate to multicistronic vectors and methods for their design. Methods and compositions of the invention include a vector including at least two cistrons, wherein a first cistron includes a first promoter and a first nucleic acid sequence encoding one or more therapeutic agents, and wherein a second cistron comprises a second promoter and a second nucleic acid sequence encoding one or more RNA molecules that interfere with the expression of a biological response modifier or the therapeutic agent, wherein the expression of the first sequence is under control of the first promoter and expression of the second sequence is under control of the second promoter. In some embodiments of the invention, the vector is a plasmid vector or a viral vector. In some embodiments, the first promoter is an operably linked promoter/enhancer sequence is an operably-linked promoter/enhancer sequence. In some embodiments, the promoter/enhancer sequence is a CMV promoter/enhancer sequence.

In some embodiments of the invention, the one or more RNA molecules that interfere with the expression of a biological response modifier is an RNAi. In some embodiments, the one or more RNA molecules that interfere with the expression of a biological response modifier is an siRNA, or an shRNA.

In some embodiments of the invention, the biological response modifier is involved in controlling or regulating an immune response, antigen processing and presentation, or gene silencing. In some embodiments, the biological response modifier involved in controlling or regulating an immune response is selected from the group consisting of: a cytokine, a chemokine, a co-stimulatory molecule, a checkpoint protein, a transcription factor, and a signal transduction molecule.

In some embodiments of the invention, the biological response modifier involved in antigen processing and presentation is selected from the group consisting of: a TAP protein, an immune proteasome, a standard proteasomes, a β₂ microglobulin, a MHC class I, and a MHC class II molecule. In some embodiments, the biological response modifier involved in gene silencing is selected from the group consisting of a DNA methylating agent, a chromatin controlling molecule, and an RNA regulating molecule.

In some embodiments of the invention, the biological response modifier involved in antigen processing and presentation is the transcription factor T-bet, STAT-1, STAT-4 or STAT-6.

In some embodiments of the invention, the biological response modifier involved in antigen processing and presentation is the cytokine IFN-α, IFN-γ, IL-10, IL-18m, IL-12 or TGF-β.

In some embodiments of the invention, the biological response modifier involved in antigen processing and presentation is the costimulatory factor CD40, B7.1 or B7.2.

In some embodiments of the invention, the biological response modifier involved in antigen processing and presentation is the checkpoint protein FOXp3, or a B7-like molecule.

In some embodiments of the invention, the antigen processing and presentation molecule is an MHC class I molecule, an MHC class I molecule, or a TAP protein.

In some embodiments of the invention, the biological response modifier involved in antigen processing and presentation is a TLR or a TLR downstream signaling molecule.

In some embodiments of the invention, the biological response modifier involved in antigen processing and presentation is the TLR downstream signaling molecule MyD88 or NFκ-B.

In some embodiments of the invention, the biological response modifier involved in antigen processing and presentation is a LAG-3 ligand.

In some embodiments of the invention, the biological response modifier involved in antigen processing and presentation is the dendritic cell activation suppressor SOCS1.

In some embodiments of the invention, the biological response modifier involved in antigen processing and presentation is the DNA methylating agent DMNT1.

In some embodiments of the invention, the one or more therapeutic agents include an immunotherapeutic agent or immunogen. In some embodiments of the invention, the one or more therapeutic agents include a gene therapeutic.

In some embodiments of the invention, the one or more therapeutic agents is an immunogen selected from the group consisting of tumor associated antigens, tumor specific antigens, differentiation antigens, embryonic antigens, cancer-testis antigens, antigens of oncogenes, mutated tumor-suppressor genes, unique tumor antigens resulting from chromosomal translocations, viral antigens, and fragments thereof. In some embodiments, the immunogen includes a tumor specific antigen or fragment thereof. In further embodiments, the therapeutic agent is a tumor antigen selected from the group consisting of Melan-A, tyrosinase, PRAME, PSMA, NYESO-1 and SSX-2. In some embodiments, the immunogen consists essentially of Melan-A₂₆₋₃₅, or its A27L analogue ELAGIGILTV (SEQ ID NO:1).

In some embodiments of the invention, the vector includes at least two cistrons, wherein a first cistron includes a first promoter and a first nucleic acid sequence encoding one or more Melan-A epitopes, and wherein a second cistron includes a second promoter and a second nucleic acid encoding one or more RNA molecules that interfere with the expression of a biological response modifier, wherein the expression of the first sequence is under control of the first promoter and expression of the second sequence is under control of the second promoter. In some embodiments, the one or more RNA molecules that interfere with the expression of a biological response modifier is a Melan-A siRNA.

In some embodiments of the invention, the vector is pSEM-U6-Melan-A (SEQ ID NO:6).

Embodiments of the invention include a method for designing a vector comprising two cistrons including the steps of placing a first promoter, a first sequence encoding one or more therapeutic agents, a second promoter, and a second sequence encoding one or more RNA molecules that interfere with the expression of a biological response modifier within the same vector, wherein the expression of the first sequence is under control of the first promoter and expression of the second sequence is under control of the second promoter.

In some embodiments of the invention, the method for designing a vector includes placing a first promoter, a first sequence encoding one or more therapeutic agents, a second promoter, and a second sequence encoding one or more agents that interfere with the expression of a biological response modifier within the same vector, wherein the expression of the first sequence is under control of the first promoter and expression of the second sequence is under control of the second promoter, and wherein the first and second promoter is selected from the group consisting of a tetracycline responsive promoter, a probasin promoter, a CMV promoter, and an SV40 promoter. In some embodiments, the vector is a plasmid vector. In further embodiments, the vector is a viral vector. In some embodiments, the vector is a plasmid vector selected from the group consisting of pSEM (SEQ ID NO:5 or SEQ ID NO:6), pBPL (SEQ ID NO:7) and pROC (SEQ ID NO:8). In some embodiments, the vector is a pSEM plasmid.

In some embodiments of the invention, the method for designing a vector further includes the step of placing an operably linked promoter/enhancer sequence in the vector. In some embodiments, the promoter/enhancer sequence is a CMV promoter.

In some embodiments of the invention, the method for designing a vector includes placing a first promoter, a first sequence encoding one or more therapeutic agents, a second promoter, and a second sequence encoding one or more RNA molecules that interfere with the expression of a biological response modifier within the same vector, wherein the expression of the first sequence is under control of the first promoter and expression of the second sequence is under control of the second promoter, and wherein the second sequence is an RNAi hairpin sequence.

In some embodiments of the invention, the method for designing a vector further includes the step of placing at least one of the group consisting of a reporter gene, a selectable marker, and an agent with immunomodulating or immunostimulating activity in the vector.

Embodiments of the invention include a mammalian cell transformed with a vector including at least two cistrons, wherein a first cistron includes a first promoter and a first nucleic acid sequence encoding one or more therapeutic agents, and wherein a second cistron includes a second promoter and a second nucleic acid encoding one or more RNA molecules that interfere with the expression of a biological response modifier or the therapeutic agent, wherein the expression of the first sequence is under control of the first promoter and expression of the second sequence is under control of the second promoter.

Embodiments of the invention include a therapeutic composition including a vector including at least two cistrons, wherein a first cistron includes a first promoter and a first nucleic acid sequence encoding one or more therapeutic agents, and wherein a second cistron includes a second promoter and a second nucleic acid encoding one or more RNA molecules that interfere with the expression of a biological response modifier or the therapeutic agent, wherein the expression of the first sequence is under control of the first promoter and expression of the second sequence is under control of the second promoter. In some embodiments, the therapeutic composition further includes a pharmaceutically acceptable carrier.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 illustrates an embodiment of the structure and construction of a bicistronic vector, in which the fragment comprising the U6 promoter and hairpin DNA sequence corresponding to GFP siRNA was inserted at restriction sites at the distal end of CMV promoter to generate pSEM-U6-GFP.

FIG. 2 shows a gel illustrating the knock-down effects of various combinations of siRNAs and bicistronic plasmids.

FIG. 3 illustrates the experimental setup for an immunization protocol involving five groups of HHD transgenic mice (n=10/group) in which various vectors (pSEM, pSEM-U6-GFP, pSEM-U6-Melan-A) were administered by direct injection into the inguinal lymph nodes (25 μg vector in 25 μl of PBS to each lymph node on day 1 and 4, followed by a second cluster of vector injections administered at day 11 and day 14, followed by injection of 1 mg/ml Melan-A₂₆₋₃₅ A27L peptide at day 34 and 37).

FIG. 4 illustrates the results of the immunization experiment (depicted in FIG. 3) as a bar graph, which shows that immunization of mice with the parent plasmid (pSEM) resulted in a detectable response in mice (7% Melan-A₂₆₋₃₅-specific CD8⁺ T cell response measured after the plasmid only immunization).

FIG. 5 shows a bar graph illustrating the average IFN-γ spot count for each of the five groups of HHD transgenic mice (n=10/group) that were administered vectors (pSEM, pSEM-U6-GFP, pSEM-U6-Melan-A) by direct injection into the inguinal lymph nodes as depicted in FIG. 3 and described in Example 3.

### DETAILED DESCRIPTION OF THE INVENTION

### Definitions

Unless otherwise noted, terms are to be understood according to conventional usage by those of ordinary skill in the relevant art.

As used herein, the term “multicistronic vector” or a “multicistronic construct” encompasses a transformable DNA sequence having at least two promoter sequences. In a multicistronic construct, each promoter sequence is operatively linked to a coding sequence to form a gene cassette, such that expression of each gene cassette results in the production of a corresponding ribonucleic acid. Accordingly, multicistronic constructs can include multiple gene cassettes. Preferred embodiments of the invention include bicistronic vectors or bicistronic constructs. In addition, references to “bicistronic” vectors or constructs are exemplary of “multicistronic” vectors or constructs and are, in some instances, interchangeable.

As used herein, the term “promoter” refers to a nucleic acid sequence that regulates expression of a nucleic acid, operably linked thereto. Such promoters are known to be cis-acting sequence elements required for transcription as they serve to bind DNA dependent RNA polymerase, which transcribes sequences present downstream thereof

As used herein, the term “operably linked” refers to a first nucleic acid molecule joined to a second nucleic acid molecule wherein the nucleic acid molecules are so arranged such that the first nucleic acid molecule affects the function and/or expression of the second nucleic acid molecule. The two nucleic acid molecules can be part of a single contiguous polynucleotide molecule and can be adjacent. For example, a promoter is operably linked to a polynucleotide of interest if the promoter modulates transcription of the linked polynucleotide molecule of interest.

The term “epitope” refers to a site on an antigen recognized by an antibody or an antigen receptor. A T-cell epitope is a short peptide derived from a protein antigen. Epitopes bind to MHC molecules and are recognized by a particular T cell. Epitopes as described in embodiments of the invention disclosed herein are molecules or substances capable of stimulating an immune response. An epitope can include, but is not limited to, a polypeptide or a nucleic acid encoding a polypeptide, wherein the polypeptide is capable of stimulating an immune response. In some embodiments, an epitope can include, but is not limited to, peptides presented on the surface of cells, the peptides being non-covalently bound to the binding cleft of class I MHC, such that they can interact with T cell receptors (TCRs).

As used herein, the term “immune epitope” refers to a polypeptide fragment that is an MHC epitope, and that is displayed on a cell in which immunoproteasomes are predominantly active. In some embodiments, “immune epitope” refers to a polypeptide containing an immune epitope according to the foregoing definition that is also flanked by one to several additional amino acids. In some embodiments, an “immune epitope” refers to a polypeptide including an epitope cluster sequence having at least two polypeptide sequences having a known or predicted affinity for a class I MHC. In some embodiments, an “immune epitope” refers to a nucleic acid that encodes an immune epitope according to any of the foregoing definitions.

As used herein, the term “housekeeping epitope” refers to a polypeptide fragment that is an MHC epitope, and that is displayed on a cell in which housekeeping proteasomes (also known as “standard proteasomes”) are predominantly active. In some embodiments, “housekeeping epitope” refers to a polypeptide containing a housekeeping epitope according to the foregoing definition that is also flanked by one to several additional amino acids. In some embodiments, a “housekeeping epitope” refers to a polypeptide including a epitope cluster sequence having at least two polypeptide sequences having a known or predicted affinity for a class I MHC. In some embodiments, a “housekeeping epitope” refers to a nucleic acid that encodes a housekeeping epitope according to any of the foregoing definitions.

As used herein, the term “liberation sequence” refers to a peptide comprising or encoding an epitope or an epitope analog, which is embedded in a larger sequence that provides a context allowing the epitope or epitope analog to be liberated by processing activities, including, for example, immunoproteasomal and housekeeping proteasomal processing, directly or in combination with N-terminal trimming or other physiologic processes

As used herein, the term “functional similarity” refers to sequences that differ from a reference sequence in an inconsequential way as judged by examination of a biological or biochemical property, although the sequences may not be substantially similar. For example, two nucleic acids can be useful as hybridization probes for the same sequence but encode differing amino acid sequences. Two peptides that induce cross-reactive CTL responses are functionally similar even if they differ by non-conservative amino acid substitutions (and thus may not be within the substantial similarity definition). Pairs of antibodies, or TCRs, that recognize the same epitope can be functionally similar to each other despite whatever structural differences exist. Testing for functional similarity of immunogenicity can be conducted by immunizing with the “altered” antigen and testing the ability of an elicited response, including but not limited to an antibody response, a CTL response, cytokine production, and the like, to recognize the target antigen. Accordingly, two sequences may be designed or engineered to differ in certain respects while retaining the same function. Such designed or engineered sequence variants of disclosed or claimed sequences are among the embodiments of the present invention.

As used herein, the term “encode” is an open-ended term such that a nucleic acid encoding a particular amino acid sequence can consist of codons specifying a polypeptide, or can also comprise additional sequences that are translatable, or whose presence is useful for the control of transcription, translation, or replication, or to facilitate manipulation of some host nucleic acid construct.

As used herein, the term “fragment,” when used in the context of antigens, refers to a portion of the antigen that is from about 10% to about 99% the length of the complete antigen, wherein the portion of the antigen includes an epitope that binds to MHC molecules and is recognized by a particular T cell. For example, a fragment of an antigen can be at least about 10%, 11%, 12%, 13%, 14%, 15%, 16%, 17%, 18%, 19%, 20%, 21%, 22%, 23%, 24%, or 25% of the length of the complete antigen. A fragment of an antigen can also be at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the length of the complete antigen.

As used herein, the term “expression cassette” refers to a polynucleotide sequence encoding a polypeptide, operably linked to a promoter and other transcription and translation control elements, including but not limited to enhancers, termination codons, internal ribosome entry sites, or polyadenylation sites. The cassette can also include sequences that facilitate moving it from one host molecule to another.

As used herein, the term “epitope cluster” refers to a polypeptide, or a nucleic acid sequence encoding it, that is a segment of a native protein sequence comprising two or more known or predicted epitopes with binding affinity for a shared MHC restriction element, wherein the density of epitopes within the cluster is greater than the density of all known or predicted epitopes with binding affinity for the shared MHC restriction element within the complete protein sequence. Epitope clusters and their uses are described in U.S. patent application Ser. No. 09/561,571, entitled “EPITOPE CLUSTERS,” filed on Apr. 28, 2000; Ser. No. 10/005,905, filed on Nov. 7, 2001; Ser. No. 10/026,066 (U.S. Patent Application Publication No. 2003-0215425), filed on Dec. 7, 2001; Ser. No. 10/895,523 (U.S. Patent Application Publication No. 2005-0130920), filed Jul. 20, 2004, and Ser. No. 10/896,325 filed Jul. 20, 2004, all entitled “EPITOPE SYNCHRONIZATION IN ANTIGEN PRESENTING CELLS,” each of which is incorporated herein by reference in its entirety.

As used herein, a “minigene” refers to a cDNA that encodes one or more polypeptide fragments for facilitating efficient processing and presentation of the epitope encoded within the nucleic acid sequence to trigger an immune response. The polypeptide fragment can be a “string of beads” array (i.e., two or more epitopes or at least one epitope and at least one epitope cluster) as disclosed in U.S. patent application Ser. No. 10/777,053 (U.S. Patent Application Publication No. 2004-0132088) entitled “EXPRESSION VECTORS ENCODING EPITOPES OF TARGET-ASSOCIATED ANTIGENS AND METHODS FOR THEIR DESIGN” filed on Feb. 10, 2004, which is incorporated herein by reference in its entirety; or an epitope cluster (as described above).

As used herein, a “target cell” refers to a cell associated with a pathogenic condition that can be acted upon by the components of the immune system, such as, for example, a cell infected with a virus or other intracellular parasite, or a neoplastic cell. In one embodiment, a target cell is a cell to be targeted by the vaccines and methods disclosed herein. A target cell according to this definition includes, but is not limited to, a neoplastic cell.

As used herein, a “Target-Associated Antigen (TAA)” refers to a protein or polypeptide present in a target cell.

As used herein, a “Tumor-Associated Antigen (TuAA)” refers to a TAA, wherein the target cell is a neoplastic cell. In some embodiments, a TuAA is an antigen associated with non-cancerous cells of the tumor such as tumor neovasculature or other stromal cells within the tumor microenvironment.

There is a need for the generation of new vaccines that can optimize immunogenicity and improve efficacy. Prior to embodiments of the invention disclosed herein, DNA vaccine therapies focused on the use of bicistronic vectors that expressed two or more therapeutic peptides or proteins, or alternatively, bicistronic vectors that encoded a therapeutic peptide/protein and an immune enhancing agent. Consequently, the bicistronic vectors were intended to elevate immune responses by providing greater levels of expression of delivered therapeutic peptide and/or providing positive regulation of immune response to the delivered peptide by expression of an immune enhancing agent. In contrast, embodiments of the invention disclosed herein provide a new class of gene vectors and methods for the design of multicistronic plasmids that co-express prophylactic agents and/or therapeutic peptides with agents that interfere with the expression of a biological response modifier. The new class of vectors is designed to improve the immunogenicity of DNA vaccines and their application as therapeutics in treating a disease or condition.

In preferred embodiments, the interfering agent encoded by the multicistronic vector embodiments is an interfering RNA. Interfering RNA embodiments, such as, for example, RNAi, have not previously been used as a component in DNA vaccines and DNA vaccine compositions. Accordingly, the use of RNAi as an interfering agent in the vectors and compositions disclosed herein represents a novel use that was not considered previously in the field. The vectors and compositions disclosed herein, provide a significant advantage in that they eliminate the need for co-injection of the interfering agent (such as, for example, siRNA) separately into a cell. In addition, the vectors and compositions disclosed herein can also specifically target antigen-presenting cells (APCs) that express an antigen of interest. While not wanting to limit the invention disclosed herein, it is believed that the bicistronic vectors disclosed herein can function as an immunotherapeutic by interfering with regulators of the immune response and/or as a gene therapeutic by inhibiting or down-regulating cellular components that are responsible for silencing gene expression or inducing apoptosis.

Vectors/Plasmids

As discussed elsewhere herein, embodiments of the invention provide a new class of vectors comprising a first sequence that encodes one or more therapeutic agents and a second sequence from which one or more agents that interfere with the expression of a biological response modifier (BRM) is expressed. In preferred embodiments, the interfering agent can be an RNAi molecule. A nucleic acid vector directing the expression of more than one protein from a single vector is known in the art as a bicistronic or multicistronic vector. A cistron is defined as a genetic unit that encodes a single polypeptide. A cistron as used herein is active in a mammalian host, and its products are directly involved in immunotherapy or gene therapy. In some embodiments, the therapeutic agent can be one or more immunogenic agents, for use in an immunotherapy. The one or more immunogenic agents can be, for example, but not limited to, an antigen, such as a tumor associated antigen. Thus, in some embodiments, the therapeutic agent can be one or more gene therapeutic agents for use in a gene therapy.

In some embodiments, one cistron can encode a therapeutic agent that is a peptide and can be, for example, but is not limited to, a Melan-A minigene. In some embodiments, a second cistron can be an agent that interferes with the expression of a BRM or a therapeutic agent such as, for example, an RNAi molecule. Therefore, in embodiments of the invention, there is provided bicistronic vectors for the treatment of a disease or condition such as, for example, but not limited to, cancer, chronic diseases, and inflammatory diseases.

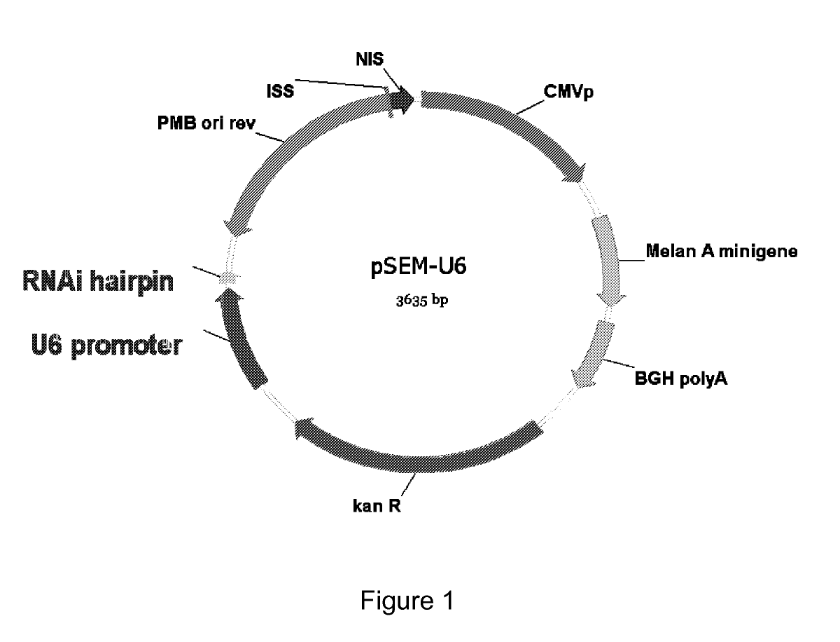

In designing the various bicistronic vector embodiments of the invention (see, for example, FIG. 1), the nucleic acid sequence (e.g. cDNA) encoding the therapeutic agent in the plasmid is placed under the control of a promoter/enhancer sequence which allows for efficient transcription of messenger RNA for the polypeptide upon uptake by a cell, such as, for example, an antigen-presenting cell (APC). Promoters that can be employed in embodiments of the invention are well known to one of ordinary skill in the art. Such promoters include, for example, viral and cellular promoters. Viral promoters can include, for example, but are not limited to, the cytomegalovirus (CMV) promoter, the major late promoter from adenovirus 2 and the SV40 promoter. Examples of cellular promoters include, for example, but are not limited to, the mouse metallothionein 1 promoter, elongation factor 1 alpha (EF1), MHC Class I and II promoter, and CD3 promoterfor T cell specific expression.

In some embodiments, control of the nucleic acid sequence from which one or more agents that interfere with the expression of biological response modifiers (BRMs) is expressed, is modeled on promoters used for expression cassettes of short hairpin RNA (shRNA). The expression cassettes of shRNA delivery vectors typically exploit RNA polymerase III (Pol III) promoters, and in some embodiments, a Pol II promoter can be used. However, the use of Pol II promoters for shRNA production is subject to certain considerations such as, for example, the need for both a very short distance (about 6 bp) between the Pol II promoter and the shRNA sequence as well as a short polyadenylation signal (Zhou et al. 2005. Nucleic Acids Res. 33, e62, which is incorporated herein by reference in its entirety); and the need for an intron between the Pol II promoter and the shRNA sequence for efficient production (Yang et al. 2004. FEBS Lett. 576: 221-225, which is incorporated herein by reference in its entirety). Preferably, the promoters used to direct the expression of shRNAs are H1 promoters, U6 promoters or CMV promoters. Other promoters that can be employed in the design of the bicistronic vectors disclosed herein can be readily determined by the skilled artisan. Particular embodiments of the invention employ a promoter/enhancer sequence from cytomegalovirus (CMVp).

In designing embodiments of the bicistronic vector disclosed herein, a bovine growth hormone polyadenylation signal (BGH polyA) at the 3′ end of the encoding sequence can be provided as a signal for polyadenylation of the messenger RNA to increase its stability as well as for translocation out of the nucleus and into the cytoplasm for translation. To facilitate plasmid transport into the nucleus after uptake, a nuclear import sequence (NIS) from simian virus 40 (SV40) can be inserted in the plasmid backbone. The plasmid design can also include immunostimulatory motifs. For example, in some embodiments, the vector (as exemplified in the pSEM-U6 plasmid in FIG. 1) can include two copies of a CpG immunostimulatory motif, one in the NIS sequence and one in the plasmid backbone.

In some embodiments, at least one further cistron in the bicistronic or multicistronic vector comprises a reporter gene. Reporter genes are well known in the art, and can facilitate the detection of cells expressing a functional protein from a vector. Detection of reporter proteins can be carried out either directly or by providing a substrate for an enzymatic reaction that produces a colored, luminescent, or fluorescent product that is readily detectable by naked eye or detector, with or without microscopy. Examples of reporter genes include genes coding for β-galactosidase, firefly luciferase, green fluorescent protein (GFP), or the red fluorescent protein from Discosoma species (DsRed). In particular embodiments, green fluorescent protein (GFP) is used as the reporter gene.

Utilizing the vector components discussed herein, some embodiments of the invention include the design and construction of a variety of bicistronic vectors that comprise RNAi such as, for example: pSEM-U6-Melan-A, pSEM-U6-T-bet, pSEM-U6-MyD88, pSEM-U6-SOCS1, pSEM-U6-DMNT1, pSEM-U6-HLA, pSEM-U6-TAPs, and pSEM-U6-FoxP3. In some embodiments, there is provided a pSEM-U6-Melan-A bicistronic vector for use as a therapeutic. In some embodiments, a recombinant DNA plasmid vaccine comprising a pSEM vector, a pROC vector, or a pBPL,vector (described in detail and referred to as pMA2M in U.S. Publication No. 20030228634, which is incorporated herein by reference in its entirety; and disclosed in U.S. Provisional Patent Application No. 60/691,579 and U.S. Publication Nos. 20030220634, each of which is incorporated herein by reference in its entirety) is employed. The pSEM plasmid, as disclosed herein encodes a polypeptide with an HLA A2-specific CTL epitope ELAGIGILTV (SEQ ID NO. 1) from Melan-A₂₆₋₃₅ A27L, and a portion (amino acids 31-96) of Melan-A (SEQ ID NO. 2) including the epitope clusters at amino acids 31-48 and 56-69. These epitope clusters were previously disclosed in U.S. patent application Ser. No. 09/561,571, entitled “EPITOPE CLUSTERS,” which is incorporated herein by reference in its entirety. Peptide analogues of Melan-A₂₆₋₃₅ A27Nva are disclosed in U.S. patent application Ser. No. 11/156,369, and U.S. Provisional Patent Application No. 60/691,889, both entitled “EPITOPE ANALOGS,” each of which is incorporated herein by reference in its entirety. The pSEM plasmid encodes the Melan-A epitopes in a manner that allows for their expression and presentation by pAPCs.

Immunotherapy Approaches

The multicistronic vectors disclosed herein have utility in immunotherapy for preventing and treating disorders, diseases, conditions and infections by inducing or enhancing or stimulating an immune responses in a subject when directed at antigens associated with such disorders, diseases, conditions and infections.

Immunotherapy can be active or passive, specific or nonspecific, depending on the process of host immune system stimulation. In some embodiments, an active immunotherapy approach is provided. The immunogenic multicistronic vectors disclosed herein allow for efficient, transient, long lasting expression of therapeutic proteins or peptides coexpressed with one or more agents that interfere with the expression of biological response modifiers, wherein the therapeutic proteins and interfering agents are encoded within the same vector and whose expression is under the control of different promoters. The one or more therapeutic proteins or peptides can include an immunogen that is selected from, but is not limited to, tumor associated antigens, tumor specific antigens, differentiation antigens, embryonic antigens, cancer-testis antigens, antigens of oncogenes, mutated tumor-suppressor genes, unique tumor antigens resulting from chromosomal translocations, viral antigens, and fragments thereof, and the like.

Immunotherapeutic multicistronic vectors can include vectors coexpressing an immunizing antigen and one or more interfering RNAs that suppress expression of molecules that regulate the immune response (such as IL-10, TGF-β, and FoxP3). Such vectors can be important for induction of strong, persisting immunity, especially in chronic infection and cancer. Other exemplary vectors include, but are not limited to, plasmids that coexpress an immunizing or tolerizing antigen and one or more siRNAs blocking pro-inflammatory pathways (STATs, T-bet, NF-κB, TLRs, IFN-α, IFN-γ). Such vectors can enable induction of therapeutic/regulatory responses or tolerance against disease associated proteins such as, for example, those involved in autoimmune diseases. In some embodiments, plasmids or other vectors can coexpress immunizing proteins and siRNA that specifically inhibit the expression of immune proteasomes, such that the activity of standard proteasomes for antigen processing becomes dominant in the APC. Such vectors can allow expression of two or more epitopes by APCs that mimic, to a greater extent, the spectrum of epitopes expressed by tumor cells and achieve epitope synchronization without requiring engineering of the native antigen sequence. These types of vectors can be used to identify epitopes that are useful for prophylaxis or therapy of cancer and other types of diseases. This type of vector strategy can also circumvent the use of cumbersome reverse immunology methods involving epitope elution from target cells or similar methods. In addition, such vectors preclude the need to use proteasome knockout mice that have more profound ontological defects. Additional vectors provided by embodiments disclosed herein, can include those that co-express a prophylactic or therapeutic protein with one or multiple RNA interfering sequences that target immune controlling molecules. Such vectors can be valuable in screening to define an optimal combination for the purpose of enhancing the beneficial effect of the vector (with application in infectious, tumoral and inflammatory disorders).

Preliminary studies suggest that a more effective CTL response can be induced using epitopes that result from processing by the housekeeping proteasome rather than by the immunoproteasome typical of the professional antigen presenting cells (pAPCs). A housekeeping epitope is an epitope produced by the proteolytic processing in cells in which the housekeeping proteasome, which is alternatively referred to as the standard or constitutive proteasome, is predominantly active. Generally, most cells express the housekeeping proteasome except for professional antigen presenting cells (pAPCs) and most cells infected with an intracellular parasite, particularly acute viral infections; and cells otherwise undergoing interferon-induced gene expression. In these cells the immune proteasome provides the predominant proteolytic processing activity, thereby establishing synchrony in the epitopes presented by both pAPCs and infected cells leading to effective immune control. However, preliminary data also suggest that cells do not strictly express immunoproteasome and that a basal level of housekeeping proteasome of about 10-20% of total proteasome is typically present in cells.

To direct, promote or force a shift from immunoproteasome activity to that of the housekeeping proteasome, a bicistronic vector of the invention can be used. A pAPC, which primarily expresses immunoproteasomal activity rather than housekeeping proteasomal activity, can be transfected with a bicistronic vector of the invention that coexpresses a tumor associated antigen and an RNAi which inhibits, decreases or abrogates the immunoproteasome activity. The pAPC thereby displays the housekeeping epitope and induces a CTL response based on the predominant expression of the housekeeping proteasome. Accordingly, in some embodiments, a bicistronic vector coexpressing an antigen and an interfering agent that inhibits immunoproteasomal activity is provided. Embodiments, of immunoproteasome inhibitors can include, but are not limited to, the X protein of the hepatitis B virus and the leaderless single chain antibodies directed against immunoproteasome-specific subunit.

Immunization with a peptide can generate a cytotoxic/cytolytic T cell (CTL) response, and attempts to further amplify this response (e.g. by additional injections) can instead lead to the expansion of a regulatory T cell population and a subsequent diminution of observable CTL activity. To control the effect of T regulatory cells on the CTL activity, in some embodiments, a bicistronic vector can be used to control or inhibit the generation and/or expansion of these cells, and thereby promote or enable the desired immune response. By introducing to an pAPC a bicistronic vector coexpressing a tumor associated antigen and a RNAi that depletes or downregulates T regulatory cells, T cell activity within a tumor or cancer can be induced, promoted, or enhanced.

The multicistronic vector embodiments can also be used to induce tolerized T cell population and/or T regulatory cells for the control of autoimmunity. In such embodiments, a bicistronic vector co-expressing an autoantigen and a RNAi that reduces or downregulates a costimulatory signal, (signal 3), or a pro-inflammatory molecule can be used to attenuate T cell activation. This can be achieved through interference with the immunological synapse, leading to the generation of T-regulatory cells and/or tolerized T cells, and/or T cells in anergy state.

In addition to the diseases and conditions discussed above, the immunogenic multicistronic compositions can be administered in treating other diseases and/or conditions in a subject. Such diseases and/or conditions can include, for example, a cell proliferative disease such as cancer. Cancers that can be treated using the immunogenic bicistronic vector composition embodiments of the invention include, for example, and in a non-limiting manner: melanoma, lung cancer including: non-small cell lung cancer (NSCLC) or small cell lung cancer (SCLC), hepatocarcinoma, retinoblastoma, astrocytoma, glioblastoma, leukemia, neuroblastoma, head and neck cancer, breast cancer, pancreatic cancer, renal cancer, bone cancer, testicular cancer, ovarian cancer, mesothelioma, cervical cancer, gastrointestinal cancer, lymphoma, colon cancer, bladder cancer and/or cancers of the blood, brain, skin, eye, tongue, gum.

The immunogenic multicistronic vector compositions disclosed herein can be used to treat cell proliferative diseases other than cancer. Other cell proliferative diseases include, for example, but are not limited to, dysplasias, pre-neoplastic lesions (e.g., adenomatous hyperplasia, prostatic intraepithelial neoplasia, cervical dysplasia, colon polyposis), or carcinoma in situ, but is not limited to such. In some embodiments of the invention, the bicistronic vector compositions disclosed herein can be used in treating a disease or condition of the neovasculature and/or of stromal cells.

Gene Therapy Approaches

In some embodiments, the multicistronic vectors disclosed herein have applicability in gene therapy. Such gene therapy vectors are applicable in suppressing a gene or genes in a target cell expressing the antigen, using, for example, interfering RNA technology. Gene therapeutic multicistronic vectors as disclosed herein allow for efficient, stable expression of therapeutic proteins coexpressed with one or more agents that interferes with the expression of biological response modifiers within the same vector but under the control of different promoters. The interference of BRM expression can lead to inhibition or down-regulation of cellular components that are responsible for silencing gene expression or inducing apoptosis.

In some embodiments, a multicistronic vector is provided, comprising a plasmid that coexpresses an immunizing protein and an interfering RNA that directly or indirectly suppresses the activity of DNA methylating enzymes. The different classes of genes that are silenced by DNA methylation include, for example, but are not limited to, tumor-suppressor genes, genes that suppress tumor invasion, and metastasis; DNA repair genes; genes for hormone receptors; and genes that inhibit angiogenesis. Such gene therapy vectors can result in a stable, longer lasting, higher level of expression of the transgene. Embodiments of the invention also include vectors that coexpress a therapeutic antigen and one or more siRNAs that inhibits, reduces or suppresses proteins in the apoptotic pathway. For example, such vectors can extend the half-life of APCs expressing an antigen of interest.

Additionally, in some embodiments, a plasmid or viral vector is provided for coexpression of a transgene and one or more inhibiting elements (e.g. a shRNA) that interfere with the dsRNA-dependent protein kinase R (PKR-dependent) machinery which plays a central role in the induction of innate immunity. Such vectors can result in a higher level and/or longer term expression of the transgene. Similarly, plasmid or viral vectors that coexpress siRNAs that interfere with class I or class II MHC expression, β2-microglobulin expression, TAP or proteasome expression are provided by embodiments disclosed herein. Such vectors expressing therapeutic transgenes, especially non-replicating viral vectors with high in vivo transduction rates, can be effective tools for gene therapy as they can circumvent mechanisms of cellular elimination by the immune system.

In some embodiments, the bicistronic gene therapy vectors disclosed herein can be used to treat diseases and conditions discussed above, such as, for example, but not limited to, cancers and inflammatory diseases.

### RNA Interference (RNAi)

Embodiments of the invention disclosed herein provide bicistronic or multicistronic vectors comprising a cistron that includes one or more agents that interfere with the expression of biological response modifiers. In embodiments where the vector acts as an immunotherapeutic agent, the one or more interfering agent(s) can be directed against expression of molecules that regulate the immune response (including, but not limited to, IL-10, TGF-β, and FoxP3). In some embodiments, the one or more interfering agent(s) can block pro-inflammatory pathways by, for example, blocking expression of molecules including, but not limited to, STATs, T-bet, NF-κB, TLRs, IFN-α, IFN-γ. In some embodiments, the one or more interfering agent(s) can specifically inhibit the expression of immune proteasomes, such that the activity of standard proteasomes for antigen processing becomes dominant in the APC. In embodiments where the vector acts as a gene therapeutic agent, the one or more interfering agent(s) can be used to inhibit or down-regulate expression of of cellular components that are responsible for silencing gene expression or inducing apoptosis. Such agents can be, for example, interfering RNAs.

RNA interference (also referred to as “RNA-mediated interference” or RNAi) is a mechanism, well known to one of ordinary skill in the art, by which suppression of specific gene expression in mammalian cells can be achieved. RNAi is a conserved process in which small interfering RNAs (siRNAs) form double-stranded structures with complementary RNA molecules and mediate their degradation. A major advantage of RNAi versus other antisense based approaches for therapeutic applications is that it utilizes cellular machinery that efficiently allows targeting of complementary transcripts, often resulting in highly potent down-regulation of gene expression. Disadvantages of RNAi include the triggering of type I interferon responses, and inefficient delivery in vivo. DNA vector-based approaches to achieve RNAi in mammalian cells can serve to overcome the obstacles of delivery in vivo. DNA-based RNAi vectors can be incorporated into viral or nonviral delivery systems.

In some embodiments, interfering RNAs or shRNAs encoding interfering RNAs can be employed to modulate the expression of biological response modifiers (biological response modifiers are discussed elsewhere, herein, in greater detail). Thus, particular embodiments provide elements, such as one or more shRNAs, siRNAs, hairpin RNAi molecules and the like, that can modulate or regulate the expression of biological response modifiers by inhibiting, silencing, reducing, down-regulating or eliminating their expression. Such RNA molecules, in an aspect of the invention, are directed against antigens, e.g., tumor associated antigens, as disclosed elsewhere herein. In some embodiments, there is provided shRNA encompassing interfering RNAs against a prophylactic and/or a therapeutic such as, for example, MART-1/Melan-A, but is not limited to such.

siRNAs can be designed so that they are specific and effective in suppressing the expression of the genes of interest. Methods of selecting the target sequences, i.e., those sequences present in the gene(s) of interest to which the siRNAs guide the degradative machinery, are directed to avoiding sequences that interfere with the siRNA's guide function while including sequences that are specific to the gene or genes. Typically, siRNA target sequences of about 19 to 23 nucleotides in length are highly effective. This length reflects the lengths of digestion products resulting from the processing of much longer RNAs (Montgomery et al., 1998).

siRNAs can be made through direct chemical synthesis; through processing of longer, double stranded RNAs through exposure to Drosophila embryo lysates; or through an in vitro system derived from S2 cells. Use of cell lysates or in vitro processing can further involve the subsequent isolation of the short (about 21-23 nucleotides) siRNAs from the lysate, etc., making the process somewhat cumbersome and expensive. Chemical synthesis proceeds by the making and annealing of two single stranded RNA-oligomers into a double stranded RNA. Methods of such chemical synthesis are diverse and well known in the art. Non-limiting examples of this methodology are provided in U.S. Pat. Nos. 5,889,136; 4,415,732; 4,458,066, and in Wincott et al. (1995), each of which is incorporated herein by reference in its entirety.

International Publication Nos. WO 99/32619 and WO 01/68836, each of which is incorporated herein by reference in its entirety, suggest that RNA for use in siRNA can be chemically or enzymatically synthesized. The enzymatic synthesis disclosed in these references is by a cellular RNA polymerase or a bacteriophage RNA polymerase (e.g. T3, T7, SP6) via the use and production of an expression construct as is known in the art (see, for example, U.S. Pat. No. 5,795,715, which is incorporated herein by reference in its entirety). The constructs disclosed therein, provide templates that produce RNAs that contain nucleotide sequences identical to a portion of the target gene. The length of identical sequences provided by these references is at least about 25 bases, and can be as many as about 400 or more bases in length. An important aspect of this reference is that the authors disclose digesting longer dsRNAs to shorter sequences of about 21-25 nucleotides in length with the endogenous nuclease complex that converts long dsRNAs to siRNAs in vivo. However, they do not describe or present data for synthesizing and using in vitro transcribed 21-25 mer dsRNAs. No distinction is made between the expected properties of chemical or enzymatically synthesized dsRNA in its use in RNA interference.

Similarly, WO 00/44914, which is incorporated herein by reference in its entirety, suggests that single strands of RNA can be produced enzymatically or by partial/total organic synthesis. Preferably, single stranded RNA is enzymatically synthesized from the PCR products of a DNA template, preferably a cloned cDNA template, and the RNA product is a complete transcript of the cDNA, which can comprise hundreds of nucleotides. WO 01/36646, which is also incorporated herein by reference in its entirety, places no limitation upon the manner in which the siRNA is synthesized, providing that the RNA can be synthesized in vitro or in vivo, using manual and/or automated procedures. This reference also provides that in vitro synthesis can be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

One challenge to be met in employing therapeutic applications of RNAi technologies is the development of systems to deliver siRNAs efficiently into mammalian cells. To that end, plasmids have been designed expressing short hairpin RNAs, or stem-loop RNA structures, driven by RNA polymerase III (pol III) promoters (Brummelkamp et al. 2002. Science 296: 550-553; Paddison et al. 2002. Genes Dev. 16: 948-958, each of which is incorporated herein by reference in its entirety). Hairpin RNAs are processed to generate siRNAs in cells and thereby induce gene silencing. Pol III promoters are advantageous because their transcripts are not necessarily post-transcriptionally modified, and because they are highly active when introduced in mammalian cells. An exemplary polymerase III (pol III) promoter employed in embodiments of the invention disclosed herein is the RNA polymerase III promoter U6.

### Biological Response Modifiers

Embodiments of bicistronic plasmids disclosed herein, include one or more agents that interfere with expression of a biological response modifier. In general, embodiments of the invention provide the use of proteins that constitute either immunological targets or deterrents of the immune response. Biological response modifiers can act in an immunosuppressive or immunostimulatory manner to modulate an immune response, for example, but not limited to, by promoting an effector response or inhibiting a T regulatory response. Biological response modifiers as disclosed for use herein can further include natural or synthetic small organic molecules which exert immune modulating effects by stimulating pathways of innate immunity.

Biological response modifiers used in embodiments disclosed herein, include, for example and in a non-limiting manner: agents that are involved in the control of an immune response such as, for example, cytokines, chemokines, co-stimulatory molecules, checkpoint proteins, transcription factors, and signal transduction elements, and the like; agents that are involved in antigen processing and presentation such as, for example, TAP 1 and TAP 2 proteins, immune or standard proteasome, β₂-microglobulin, and MHC class I or II molecules, and the like; agents that are involved in regulating the apoptotic pathway; agents that are involved in gene control or silencing such as, for example, DNA methylating enzymes, chromatin controlling molecules and RNA regulating molecules, and the like. For example, cellular receptors, cytokine receptors, chemokine receptors, signal transduction elements, or transcriptional regulators can be used as BRMs in the context described herein.

In some embodiments, biological response modifiers can include, for example and in a non-limiting manner, molecules that trigger cytokine or chemokine production, such as ligands for Toll-like receptors (TLRs), peptidoglycans, LPS or analogues, imiquimodes, unmethylated CpG oligodeoxynuclotides (CpG ODNs); dsRNAs such as bacterial dsDNA (which contains CpG motifs) and synthetic dsRNA (polyl:C) on APC and innate immune cells that bind to TLR9 and TLR3, respectively.

One class of biological response modifiers considered useful in embodiments of the invention disclosed herein, includes small organic natural or synthetic molecules, which exert immune modulating effects by stimulating pathways of innate immunity. It has been shown that macrophages, dendritic and other cells carry so-called Toll-like receptors (TLRs), which recognize pathogen-associated molecular patterns (PAMPs) on micro-organisms (Thoma-Uszynski, S. et al., Science 291:1544-1547, 2001; Akira, S., Curr. Opin. Immunol., 15: 5-11, 2003; each of which is incorporated herein by reference in its entirety). Furthermore, in some embodiments, small molecules that bind to TLRs can be used, such as a new generation of purely synthetic anti-viral imidazoquinolines, e.g., imiquimod and resiquimod, that have been found to stimulate the cellular path of immunity by binding the TLRs 7 and 8 (Hemmi, H. et al., Nat Immunol 3: 196-200, 2002; Dummer, R. et al., Dermatology 207: 116-118, 2003; each of which is incorporated herein by reference in its entirety).

Biological response modifiers that interact directly with receptors that detect microbial components can also be used in designing a bicistronic vector of the invention. Additionally, molecules that act downstream in the signalling pathway can be used. Antibodies that bind to co-stimulatory molecules (such as, for example, anti-CD40, CTLA-4, anti-OX40, and the like) are also useful in embodiments of the invention. In some embodiments, biological response modifiers employed can include, for example, but not limited to, IL-2, IL-4, TGF-β, IL-10, IFN-γ and the like; or molecules that trigger their production. Other biological response modifiers, can include, for example, but not limited to, cytokines such as IL-12, IL-18, GM-CSF, flt3 ligand (flt3L), interferons, TNF-α, and the like. Additionally, chemokines, such as, for example, but not limited to, IL-8, MIP-3α, MIP-1α, MCP-1, MCP-3, RANTES, and the like can also be employed in embodiments of the invention disclosed herein.

In addition, biological response modifiers can include co-stimulatory molecules such as, but not limited to, B7 molecules which stimulate T cell proliferation. The interfering agent (e.g. RNAi) can interfere with proinflammatory cytokines such as IL-6, IL-12, IL-18, IFN-alpha, and IFN-gamma and the like.

In some embodiments, biological response modifiers can include a costimulatory signal, (signal 3), or a pro-inflammatory molecule that affects T cell activation. An interfering agent directed against such BRMs can interfere with the immunological synapse, leading to the generation of T-regulatory cells and/or tolerized T cells, and/or T cells in anergy state.

### Therapeutic Agents

In using therapeutic DNA vaccines for treating or eradicating a disease or condition, an antigen is preferably acquired and processed into peptides that are subsequently presented on class I MHC-peptide complexes located on the pAPC surface in order to stimulate a CTL response. CTLs are thereby induced to proliferate and recirculate through the body in search of the target diseased cells with similar class I MHC-peptide complexes on their surface. Cells presenting these complexes are then destroyed by the cytolytic activity of the CTL. If the target diseased cell does not express the predominantly expressed proteasome expressed by a pAPC, then the epitopes may not be “synchronized” and CTL can fail to find the desired peptide target on the surface of the diseased cell.

It is desirable, therefore, to consider and account for the Class I MHC-peptide complex present on the target tissue when designing effective DNA vaccines. That is, effective antigens used to stimulate CTL to attack the target diseased tissue are those that are naturally processed and presented on the surface of the diseased tissue. For tumors and chronic infection, this generally means that the CTL epitopes are those that have been processed by the housekeeping proteasome. To generate an effective therapeutic vaccine, CTL epitopes are identified based on the knowledge that such epitopes are produced by the housekeeping proteasome system. Once identified, these epitopes, embodied as peptides or products expressed by appropriately encoded nucleic acid vectors, can be used to successfully immunize or induce therapeutic CTL responses against housekeeping proteasome expressing target cells in the host.

In designing DNA vaccines, an additional aspect to consider is that the immunization with DNA requires that APCs take up the DNA and express the encoded proteins or peptides. Therefore, upon immunization with a generated vector, APCs can be stimulated to express an epitope which is then displayed on class I MHC on the surface of the cell for stimulating an appropriate CTL response.

To evaluate the importance of plasmid-driven antigen expression within the lymph node, and to study whether priming is exclusively caused by activation of innate immunity via plasmid-TLR interaction, experimental studies were conducted to examine the effect, if any, of specific RNA interference of MART-1/Melan-A expression on induction of the immune response. Accordingly, an embodiment of the novel bicistronic vector that co-expresses the antigen and a shRNA encompassing RNAi against MART-1/Melan-A has been designed and administered.

In designing a bicistronic vector as disclosed herein, embodiments of the invention also provide prophylactic or therapeutic proteins co-expressed with agents that interfere with the expression of biological response modifiers. In some embodiments, antigens can be used as therapeutic agents and can be coexpressed with agents that interfere with the expression of biological response modifiers. The antigens used in embodiments of the invention can include, but are not limited to, proteins, peptides, polypeptides and derivatives thereof, and can also be non-peptide macromolecules.

In embodiments of the invention, an antigen is one that stimulates the immune system of a subject having a malignant tumor or infectious disease to attack the tumor or pathogen, thereby inhibiting its growth or eliminating it, and hence treating or curing the disease. The antigen, in some instances, can be matched to the specific disease found in the subject being treated, to induce a CTL response (also referred to as a cell-mediated immune response), thereby eliciting a cytotoxic reaction by the immune system that results in lysis of target cells (e.g., the malignant tumor cells or pathogen-infected cells).

Embodiments of the invention can also utilize peptide antigens of about 8-15 amino acids in length. Such a peptide can be an epitope of a larger antigen, i.e., a peptide having an amino acid sequence corresponding to a site on the larger antigen that is presented by MHC/HLA molecules and can be recognized, for example, by an antigen receptor or T-cell receptor. Such peptide antigens are available to one of skill in the art and are disclosed, for example, in U.S. Pat. Nos. 5,747,269 and 5,698,396; International Application No. PCT/EP95/02593, filed Jul. 4, 1995; and International Application No. PCT/DE96/00351, filed Feb. 26, 1996, each of which is incorporated herein by reference in its entirety. Additional epitopes, as well as methods of epitope discovery, are described, for example, in U.S. Pat. Nos. 6,037,135 and 6,861,234, each of which is incorporated herein by reference in its entirety.

While in the general case the antigen ultimately recognized by a T cell is a peptide, the form of antigen actually administered as the immunogenic preparation need not be a peptide per se. When administered, the epitopic peptide or peptides can be included within a longer polypeptide, which can be, for example, a complete protein antigen or a segment thereof, or an engineered sequence that has functional similarity to such. Engineered sequences can include, for example, polyepitopes and epitopes incorporated into a carrier sequence, such as an antibody or viral capsid protein. Such longer polypeptides can include epitope clusters, such as, for example, those described in U.S. patent application Ser. No. 09/561,571 entitled “EPITOPE CLUSTERS,” which is incorporated herein by reference in its entirety. The epitopic peptide, or the longer polypeptide in which it is included, can be a component of a microorganism (e.g., a virus, bacterium, protozoan, etc.), or a mammalian cell (e.g., a tumor cell or antigen presenting cell), or a lysate, including whole or partially purified lysates, of any of the foregoing. The epitopic peptide, or the longer polypeptide in which it is included, can be used as complexes with other proteins, for example, heat shock proteins. In some embodiments, the epitopic peptide, or the longer polypeptide in which it is included, can be covalently modified, such as, for example, by lipidation. Alternatively, the epitopic peptide, or the longer polypeptide in which it is included, can be made as a component of a synthetic compound, such as, for example, dendrimers, multiple antigen peptides systems (MAPS), and polyoximes. In some embodiments, the epitopic peptide, or the longer polypeptide in which it is included, can be incorporated into liposomes or microspheres, etc. As used herein, the term “polypeptide antigen” encompasses all such possibilities and combinations.

Embodiments of the invention provide that the antigen can be a native component of the microorganism or mammalian cell. The antigen can also be expressed by the microorganism or mammalian cell through recombinant DNA technology or, in the case of antigen presenting cells (APCs), by pulsing or loading the cell with polypeptide antigen prior to administration. Additionally, the antigen can be administered as a nucleic acid that encodes the antigen such that the antigen is subsequently expressed by a cell after administration of the nucleic acid to the cell. Finally, whereas the classical class I MHC molecules present peptide antigens, additional class I molecules can be adapted to present non-peptide macromolecules. Exemplary non-peptide macromolecules include, but are not limited to, lipids and glycolipids. As used in herein, the term “antigen” includes such macromolecules as well. Moreover, a nucleic acid-based vaccine can encode one or more enzymes for the synthesis of such a macromolecule and thereby facilitate antigen expression of the macromolecule on an APC. In some embodiments, the nucleic acid-based vaccine can encode two, three, four or five enzymes for synthesis and antigen expression of the macromolecule on an APC.

Other therapeutic or prophylactic proteins useful in embodiments of the invention include, for example: tumor specific antigens, differentiation antigens, embryonic antigens, cancer-testis antigens, antigens of oncogenes, mutated tumor-suppressor genes, unique tumor antigens resulting from chromosomal translocations, viral antigens, and any other antigen that is presently apparent or will be in the future to one of skill in the art. Additional antigens that can be employed in embodiments of the invention include, for example, those found in infectious disease organisms, such as structural and non-structural viral proteins.

In light of the aforementioned, antigens useful in embodiments of the invention, include tumor-specific antigens (TSAs) or tumor-associated antigens (TuAAs). A TSA is unique to tumor cells and does not occur on other cells in the body. TuAAs are TAAs, wherein the target cell is a neoplastic cell. TuAAs can be antigens that are expressed on normal cells during fetal development when the immune system is immature and unable to respond, or they can be antigens that are normally present at extremely low levels on normal cells but are expressed at much higher levels on tumor cells. In some embodiments, a TuAA is an antigen associated with non-cancerous cells of the tumor, such as, for example, tumor neovasculature or other stromal cells within the tumor microenvironment.

In some embodiments, the antigen can be an autoantigen, such as, for example, but not limited to, insulin, GAD65, or HSP for treatment of Type 1 diabetes. In some embodiments, the autoantigen can be, but is not limited to, myelin basic protein (MBP), proteolipid protein (PLP), or myelin oligodendrocyte glycoprotein (MOG) for treatment of multiple sclerosis.

In some embodiments of the invention, the TuAA Melan-A, also known as MART-1 (Melanoma Antigen Recognized by T cells) is employed. Melan-A/MART-1 is a melanin biosynthetic protein expressed at high levels in melanomas. Melan-A/MART-1 is well known in the art and is disclosed in U.S. Pat. Nos. 5,994,523; 5,874,560; and 5,620,886, each of which is incorporated herein by reference in its entirety. A preferred embodiment provides the Melan-A TuAA, Melan-A₂₆₋₃₅, represented herein by SEQ. ID NO: 1. Non-limiting examples of other TuAAs that are useful in embodiments of the invention include tyrosinase, SSX-2, NY-ESO-1, PRAME, and PSMA (prostate-specific membrane antigen). The TuAAs useful in embodiments of the invention disclosed herein can comprise the native sequence or analogues thereof, such as those disclosed in U.S. Provisional Patent Application No. 60/691,889; U.S. patent application Ser. Nos. 11/455,278, 11/454,633, and 11/454,300; and PCT Patent Application No. PCT/US2006/023489; and U.S. Patent Application Publication Nos. 20060057673 and 20060063913; each of which is incorporated herein by reference in its entirety.

Additional peptides, and peptide analogues that can be employed in embodiments of the invention are disclosed in U.S. Patent Application No. 60/581,001, filed on Jun. 17, 2004 entitled SSX-2 PEPTIDE ANALOGS; and 60/580,962 entitled NY-ESO PEPTIDE ANALOGS; U.S. patent application Ser. No. 09/999,186, filed Nov. 7, 2001, entitled METHODS OF COMMERCIALIZING AN ANTIGEN; U.S. patent application Ser. No. 11/323,572 filed on Dec. 29, 2005, entitled, METHODS TO ELICIT, ENHANCE AND SUSTAIN IMMUNE RESPONSES AGAINST MHC CLASS I-RESTRICTED EPITOPES, FOR PROPHYLACTIC OR THERAPEUTIC PURPOSES; and U.S. patent application Ser. No. 11/323,520 filed on Dec. 29, 2005, entitled METHODS TO BYPASS CD4⁺ CELLS IN THE INDUCTION OF AN IMMUNE RESPONSE, each of which is hereby incorporated by reference in its entirety. Beneficial epitope selection principles for immunotherapeutics are disclosed in U.S. patent application Ser. No. 09/560,465 (filed on Apr. 28, 2000), Ser. No. 10/026,066 (filed on Dec. 7, 2001; Publication No. 20030215425 A1), and Ser. No. 10/005,905 (filed on Nov. 7, 2001) all entitled EPITOPE SYNCHRONIZATION IN ANTIGEN PRESENTING CELLS; Ser. No. 09/561,571 (filed on Apr. 28, 2000) entitled EPITOPE CLUSTERS; Ser. No. 10/094,699 (filed on Mar. 7, 2002; Publication No. 20030046714 A1) entitled ANTI-NEOVASCULATURE PREPARATIONS FOR CANCER; and Ser. No. 10/117,937 (filed on Apr. 4, 2002; Publication No. 20030220239 A1) and Ser. No. 10/657,022 (filed on Sep. 5, 2003; Publication No. 20040180354 A1), and PCT Application No. PCT/US2003/027706 (Publication No. WO/04022709A2) all entitled EPITOPE SEQUENCES, and U.S. Pat. No. 6,861,234; each of which is hereby incorporated by reference in its entirety.

In some embodiments, additional antigens that can be employed include, for example and in a non-limiting manner: gp100 (Pmel 17), TRP-1, TRP-2, MAGE-1, MAGE-3, BAGE, GAGE-1, GAGE-2, CEA, RAGE, SCP-1, Hom/Mel-40, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CAM 17.1, NuMa, K-ras, β-Catenin, CDK4, Mum-1, p16, p15, 43-9F, 5T4, 791Tgp72, alpha-fetoprotein, β-HCG, BCA225, BTAA, CA 125, CA 15-3\CA 27.29\BCAA, CA 195, CA 242, CA-50, CAM43, CD68\KP1, CO-029, FGF-5, G250, Ga733\EpCAM, HTgp-175, M344, MA-50, MG7-Ag, MOV18, NB/70K, NY-CO-1, RCAS1, SDCCAG16, PLA2, TA-90\Mac-2 binding protein\cyclophilin C-associated protein, TAAL6, TAG72, TLP, and TPS. These protein-based antigens are known and available to the skilled artisan, both in the literature and/or commercially.

Additional therapeutic molecules useful in some embodiments of the invention include, but are not limited to, transcription factors such as T-bet, STAT-1 STAT-4 and STAT-6. In some embodiments of the invention, the targeted molecules can include TLR and its downstream signaling molecules such as, for example, but not limited to, MyD88, NFκ-B, and the like. Cytokines are also useful in embodiments of the invention, such as, for example, but not limited to, G-CSF, GM-CSF, IFN, IFN-α, IFN-β, IFN-γ, IL-2, IL-3, IL-4, IL-8, IL-9, IL-10, IL-12, IL-13, IL-14, IL-15, IL-18, TNF, TGF-α, TGF-β and the like. Costimulatory factors such as, CD40 B7.1 and B7.2 are also useful in some embodiments. In some embodiments, checkpoint proteins such as, for example, but not limited to, FOXp3, B7-like molecules, LAG-3 ligands and such molecules can be used. Proteins present in the antigen presentation pathway such as, for example, but not limited to, HLA and TAPs (Transporters associated with Antigen Processing-1 and -2 (TAP1 and TAP2)) can also be used in embodiments of the invention. Dendritic cell activation suppressor SOCS1 and proteins in the DNA methylation pathway such as DMNT1 can also be used in embodiments disclosed herein. Proteins present in the apoptotic pathway can also be used in embodiments disclosed herein. Embodiments of the invention can employ one or more of the molecules disclosed herein, alone or in various combinations, when designing a bicistronic vector of the invention.

Any antigen disclosed herein, can be linked as a string-of-bead arrays or polyepitopes for use in the design of a bicistronic vector. String-of-bead arrays or polyepitopes are well known in the art as disclosed in, for example, in International Publication No. WO 01/19408A1; WO 99/55730A2; WO 00/40261A2; WO 96/03144A1; WO 01/23577A3; WO 97/41440A1; WO 98/40500A1, WO 01/18035A2, WO 02/068654A2; WO 01/58478A; WO 01/11040A1; WO 01/89281A2; WO 00/73438A1; WO 00/71158A1; WO 00/52451A1; WO 00/52157A1; WO 00/29008A2; WO 00/06723A1 and U.S. Pat. Nos. 6,074,817; 5,965,381; 6,130,066; 6,004,777; 5,990,091; each of which is incorporated herein by reference in its entirety.

In some embodiments, new peptides identified by the method disclosed in U.S. Pat. No. 6,861,234, entitled “METHOD OF EPITOPE DISCOVERY” and U.S. patent application Ser. No. 10/026,066 (Publication No. 2003-0215425) filed on Dec. 7, 2000 and entitled “EPITOPE SYNCHRONIZATION IN ANTIGEN PRESENTING CELLS,” (each of which is hereby incorporated by reference in its entirety) that are presently apparent or will be apparent in the future to one of ordinary skill in the art, can be used in embodiments disclosed herein.

Additional exemplary peptides that can be used as therapeutic peptides include those disclosed in Tables 1A-1C of WO 02/081646 (which is incorporated herein by reference in its entirety) as well as those disclosed in Tables 1A and 1B of WO 04/022709 (which is incorporated herein by reference in its entirety).

### Methods of Delivering Compositions

In some embodiments, the preferred administration of the bicistronic vectors, comprising one or more therapeutic proteins coexpressed with one or more agents that interfere with expression of biological response modifiers, is via lymph node injection. Lymph node injection is preferred as it allows for direct delivery into the organs where the immune responses are initiated and amplified according to an optimized immunization schedule.

To introduce an immunogenic bicistronic vector composition as disclosed herein into the lymphatic system of the patient, the composition is preferably directed to a lymph vessel, lymph node, the spleen, or other appropriate portion of the lymphatic system. An advantage of the bicistronic vectors disclosed herein is that these vectors can obviate the need for separate injections of the therapeutic molecules of interest. In embodiments of the invention, the bicistronic vector can be used in a prime/boost protocol (as disclosed in U.S. Patent Application 60/831,256 entitled “METHOD TO ELICIT, ENHANCE AND SUSTAIN IMMUNE RESPONSES AGAINST MHC CLASS-I RESTRICTED EPITOPES, FOR PROPHYLACTIC OR THERAPEUTIC PURPOSES,” which is incorporated herein by reference in its entirety) wherein the bicistronic vector composition is injected into the inguinal lymph node followed by a subsequent administration of a peptide antigen as a bolus. In some embodiments, one or more components can be delivered by infusion, generally over several hours to several days. Preferably, the composition is directed to a lymph node such as an inguinal or axillary node by inserting a catheter or needle to the node and maintaining the catheter or needle throughout the delivery. Suitable needles or catheters are available that are made of metal or plastic (e.g., polyurethane, polyvinyl chloride (PVC), TEFLON, polyethylene, and the like). In inserting the catheter or needle into the inguinal node for example, the inguinal node is punctured under ultrasonographic control using a Vialon™ Insyte W™ cannula and catheter of 24G3/4 (Becton Dickinson, USA) which is fixed using Tegaderm™ transparent dressing (Tegaderm™, St. Paul, Minn., USA); this procedure is generally performed by an experienced radiologist. The location of the catheter tip inside the inguinal lymph node is confirmed by injection of a minimal volume of saline, which immediately and visibly increases the size of the lymph node. The latter procedure allows confirmation that the tip is inside the node. This procedure can be performed to ensure that the tip does not slip out of the lymph node and can be repeated on various days after implantation of the catheter. In the event that the tip does slip out of location inside the lymph node, a new catheter can be implanted.

The therapeutic compositions disclosed herein can be administered to a patient in a manner consistent with standard vaccine delivery protocols that are well known to one of ordinary skill in the art. Methods of administering immunogenic bicistronic vector composition embodiments of the present invention comprising one or more prophylactic or therapeutic agent with one or more agent that interfere with the expression of biological response modifiers include, without limitation: transdermal, intranodal, perinodal, oral, intravenous, intradermal, intramuscular, intraperitoneal, mucosal administration, and delivery by injection or instillation or inhalation. Particularly useful methods of vaccine delivery to elicit a CTL response are disclosed in Australian Patent No. 739189; U.S. Pat. Nos. 6,994,851 and 6,977,074 both entitled “A METHOD OF INDUCING A CTL RESPONSE,” each of which is incorporated herein by reference in its entirety.

It is useful to consider various parameters in delivering or administering a bicistronic vector immunogenic composition to a subject. In addition, a dosage regimen and immunization schedule can be employed. Generally, the amount of the components in the therapeutic composition will vary from patient to patient, from therapeutic agent to therapeutic agent, and from biological response modifier to biological response modifier, depending on such factors as: the activity of the therapeutic agent or biological response modifier in inducing a response; the flow rate of the lymph through the patient's system; the weight and age of the subject; the type of disease and/or condition being treated; the severity of the disease or condition; previous or concurrent therapeutic interventions; the capacity of the individual's immune system to synthesize antibodies; the degree of protection desired; the manner of administration and the like, all of which can be readily determined by the skilled practitioner.

Generally, the therapeutic compositions of the invention can be delivered at a rate of from about 1 to about 500 microliters/hour or about 24 to about 12,000 microliters/day. The concentration of the therapeutic composition is such that about 0.1 micrograms to about 10,000 micrograms of the therapeutic composition will be delivered during a 24 hour period. The flow rate is based on the knowledge that, in each minute, approximately about 100 to about 1000 microliters of lymph fluid flows through an adult inguinal lymph node. An objective is to maximize local concentration of vaccine formulation in the lymph system. A certain amount of empirical investigation on patients is conducted to determine the most efficacious level or optimal level of infusion for a given vaccine preparation in humans.

In one embodiment, the immunogenic composition disclosed herein can be administered as a plurality of sequential doses. Such plurality of doses can be 2, 3, 4, 5, 6 or more doses as is found effective. In some embodiments, the doses of the immunogenic bicistronic compositions disclosed herein are administered within about weeks or days of each other and/or of a peptide boost into the right or left inguinal lymph nodes. It can be desirable to administer the plurality of doses of the immunogenic bicistronic vector composition and/or of a peptide boost of the invention at an interval of days, where several days (1, 2, 3, 4, 5, 6, or 7, or more days) lapse between subsequent administrations. In other instances, it can be desirable for subsequent administration(s) of the compositions of the invention to be administered via bilateral inguinal lymph node injection within about 1, 2, 3, or more weeks or within about 1, 2, 3, or more months following the initial dose administration.

Administration can be in any manner compatible with the dosage formulation and in such amount as will be therapeutically effective. An effective amount or dose of immunogenic composition embodiments of the present invention is that amount found to provide a desired response in the subject to be treated.

### Kits

Any of the compositions described herein can be assembled together in a kit. More particularly, all or a subset of the components for designing and constructing bicistronic vector embodiments of the present invention can be packaged together in a kit. The one or more therapeutic agent and the one or more coexpressed agent that interfere with the expression of biological response modifiers can be packaged separately or together. In some embodiments, it is preferable to package the plasmid together with the one or more therapeutic agents or the one or more coexpressed agents that interfere with the expression of biological response modifiers. In embodiments of the invention, the therapeutic proteins, peptides, polypeptides, epitopes or nucleic acid encoding such can be packaged together, or as single molecules, or as a set of molecules. In some embodiments, the one or more coexpressed agents that interfere with the expression of biological response modifiers can be packaged together, or as single molecules, or as a set of molecules. In some embodiments, the one or more therapeutic molecules and the one or more coexpressed agents that interfere with the expression of biological response modifiers can be packaged together in a kit. Alternatively, the compositions disclosed herein can be packaged and sold individually along with instructions, in printed form or on machine-readable media, describing how they can be used in conjunction with each other to design and construct a bicistronic vector, as disclosed herein, for use as a therapeutic.

In a non-limiting example, one or more agents or reagents for designing or constructing a gene therapy vector as disclosed herein can be provided in a kit alone, or in combination with additional agents or reagents for treating a disease or condition, such as cancer. However, these components are not meant to be limiting. In some embodiments, the kits will provide a suitable container means for storing and dispensing the agents or reagents.

In some embodiments, the kit can contain, in a suitable container means, one or more therapeutic molecules and/or one or more agents that interfere with the expression of biological response modifiers and a vector such as, for example, a pSEM plasmid and instructions for designing and constructing a bicistronic vector. In one embodiment, the kit can have a single container means, and/or it can have distinct container means for additional compounds such as an immunological/therapeutic effective formulation of one or more therapeutic agents for treating a disease or condition due to, for example, a proliferative disease such as cancer. In some embodiments, the kit can further contain, in suitable container means, the one or more coexpressed agents that interfere with the expression of biological response modifiers, each in a separate container means or as a set in a single container means.

Where the components of the kit are provided in one or more liquid solutions, the liquid solution is an aqueous solution, with a sterile aqueous solution being particularly preferred. The compositions can also be formulated as a deliverable and/or injectable composition. In such embodiments, the container means can itself be a syringe, pipette, and/or other such apparatus, from which the formulation can be delivered or injected into a subject, and/or even applied to and/or mixed with the other components of the kit. In some embodiments, the components of the kit can be provided as dried powder(s). When components (e.g., reagents) are provided as a dry powder, the powder can be reconstituted by the addition of a suitable solvent. It is envisioned that the solvent can also be provided in another container means.

In some embodiments, the plasmid can be sold together with the prophylactic or therapeutic protein, peptide, epitope or nucleic acid encoding such and/or the agent(s) that interfere with the expression of biological response modifiers. In some embodiments, sets of prophylactic or therapeutic proteins, peptides, epitopes or nucleic acids encoding such can be sold together without the plasmid. Sets of a molecule corresponding to the agent that interferes with the expression of biological response modifiers can be sold together without the plasmid.

The container means will generally include at least one vial, test tube, flask, bottle, syringe and/or other container means, into which the bicistronic vector comprising: one or more prophylactic or therapeutic agents and one or more agents that interfere with the expression of biological response modifiers can be placed. The kit can also comprise a second container means for containing a sterile, pharmaceutically acceptable buffer and/or other diluent. In some embodiments, the kit can also include a means for containing the materials for practicing the methods disclosed herein, and any other reagent containers in close confinement for commercial sale. Such containers can include, for example, injection or blow-molded plastic containers into which the desired vials are retained. Irrespective of the number or type of containers, the kit(s) of the invention can also comprise, or be packaged with, an instrument for assisting with the injection/administration of the bicistronic vector comprising: one or more prophylactic or therapeutic agents and one or more agents that interfere with the expression of biological response modifiers, within the body of a subject. Such an instrument can be, for example, but not limited to, a syringe, pump and/or any such medically approved delivery vehicle.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Design and Construction of Bicistronic Vectors Co-Expressing Immunogene and RNAi

The structure and construction of pSEM plasmid (also known as pMA2M) has been previously disclosed (US Patent Application Publication 20030228634 and PCT Patent Publication WO 03/063770). Briefly, the pSEM plasmid encodes one polypeptide with an HLA A2-specific CTL epitope ELAGIGILTV (SEQ ID NO. 1) from Melan-A₂₆₋₃₅ A27L, and a portion (amino acids 31-96) of Melan-A (SEQ ID NO. 2) including the epitope clusters at amino acids 31-48 and 56-69. These clusters were previously disclosed in U.S. patent application Ser. No. 09/561,571, filed Apr. 28, 2000 entitled EPITOPE CLUSTERS, which is incorporated herein by by reference in its entirety. Flanking the defined Melan-A CTL epitope are short amino acid sequences derived from human tyrosinase (SEQ ID NOs: 3 and 4) to facilitate liberation of the Melan-A housekeeping epitope by processing by the immunoproteasome. In addition, these amino acid sequences represent potential CTL epitopes themselves. The cDNA sequence for the polypeptide in the plasmid is under the control of promoter/enhancer sequence from cytomegalovirus (CMVp), which allows efficient transcription of messenger for the polypeptide upon uptake by APCs. The bovine growth hormone polyadenylation signal (BGH polyA) at the 3′ end of the encoding sequence provides a signal for polyadenylation of the messenger to increase its stability as well as for translocation out of nucleus into the cytoplasm for translation. To facilitate plasmid transport into the nucleus after uptake, a nuclear import sequence (NIS) from simian virus 40 (SV40) has been inserted in the plasmid backbone. The plasmid carries two copies of a CpG immunostimulatory motif, one in the NIS sequence and one in the plasmid backbone. Lastly, two prokaryotic genetic elements in the plasmid are responsible for amplification in E. coli, the kanamycin resistance gene (Kan R) and the pMB1 bacterial origin of replication.

PCR reaction was performed to amplified the fragment for U6 promoter and the hairpin DNA sequence corresponding to GFP siRNA using pSilencer (Invitrogen) as the template. The resulting fragment was ligated between BspH I and BstE I sites at the distal end of CMV promoter to generate pSEM-U6-GFP to be used as a control for off-target effect of RNAi (FIG. 1). Subsequently, the sequence corresponding to siRNA for Melan-A and other targeted molecules were used to substitute sequence corresponding to hairpin for GFP siRNA, resulting in the generation of plasmid pSEM-U6-Melan-A to be used as an internal control for RNAi. The sequences of the above-mentioned two plasmids, pSEM-U6-GFP and pSEM-U6-Melan-A are disclosed as SEQ ID NO.5 and SEQ ID NO.6, respectively.

### Example 2

### In Vitro Knock Down in an Overexpression System

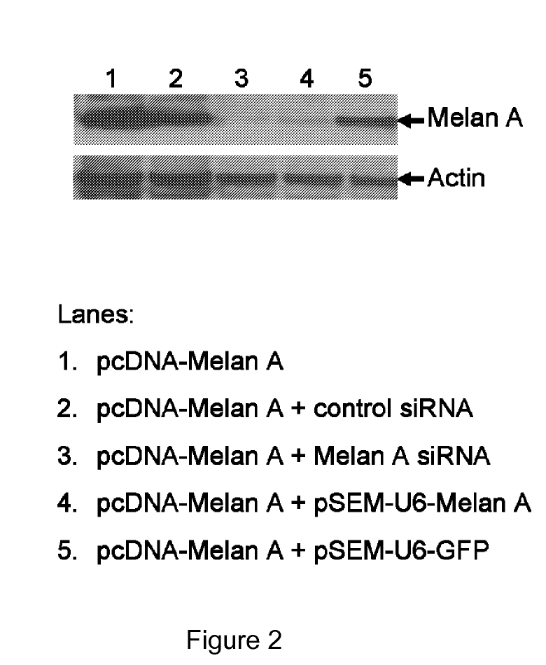

HEK 293T cells were transfected with a Melan-A-expressing plasmid pcDNA-Melan-A alone, or co-transfected with pSEM-U6-Melan-A, pSEM-U6-GFP, siRNA for Melan-A, or control siRNA, respectively. Forty-eight hours post transfection, cells were harvested and cell lysates were prepared and subjected to SDS-PAGE and immunoblot. The knock down effects of various siRNAs and bicistronic plasmids were evaluated (FIG. 2). Co-transfection of siRNA specific for Melan-A resulted in a significant decrease in the level of Melan-A expression in transfected cells, with the knock down effect being over 90%. In cells co-transfected with pcDNA-Melan-A and pSEM-U6-Melan-A, the knock down effect on Melan-A expression is estimated to be between 80-90%. A slight reduction in Melan-A expression level was also observed in samples from cells co-transfected with Melan-A-expressing plasmid and pSEM-U6-GFP, or control siRNA, respective.

### Example 3

### In Vivo Knock Down of Antigen Expression Leads to an Abolished Immune Response

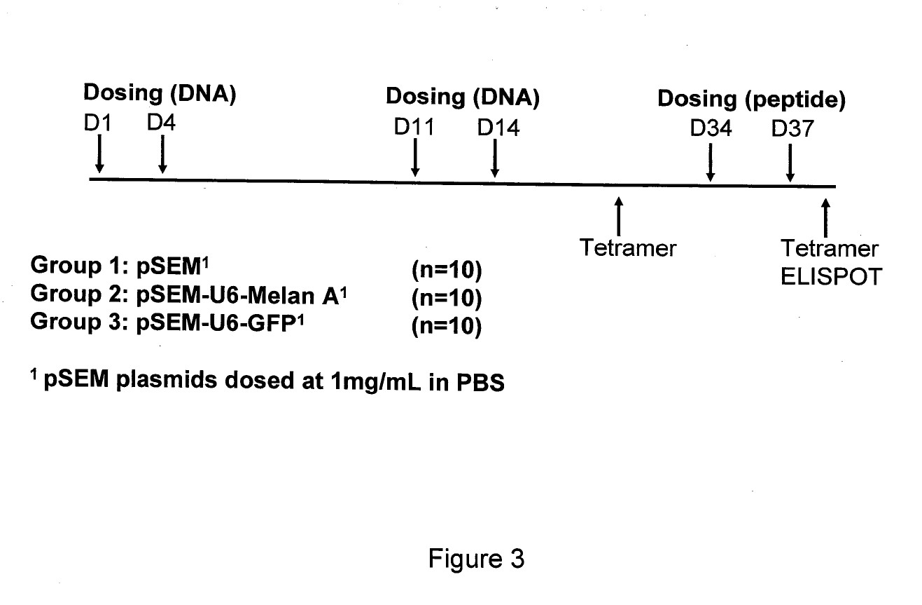

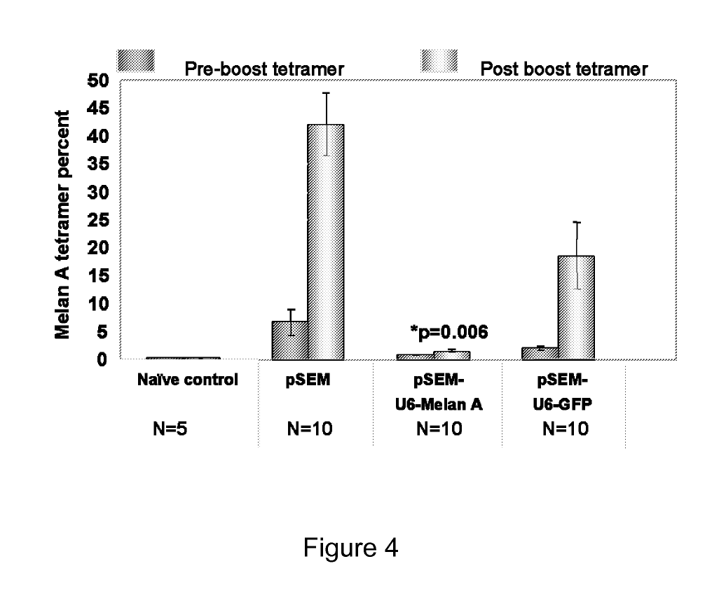

Five groups of HHD transgenic mice (n=10/group) were immunized with plasmids (pSEM, pSEM-U6-GFP, pSEM-U6-Melan-A) by direct injection into the inguinal lymph nodes of 25 μg in 25 μl of PBS to each lymph node on day 1 and 4. Mice received a second cluster of DNA injections ten days after, at day 11 and day 14, and injection of Melan-A₂₆₋₃₅ A27L peptide (1 mg/ml) at day 34 and 37 (FIG. 3). Peripheral blood was isolated from individual mice via retro-orbital bleed and mononuclear cells were separated from red blood cells following density centrifugation (Lympholyte Mammal, Cedarlane Labs). The specific CTL response in immunized animals was quantified by co-staining mononuclear cells with HLA-A2.1 MART-1₂₆₋₃₅ (ELAGIGILTV)-APC, and FITC conjugated rat anti-mouse CD8a (Ly-2) monoclonal antibody (BD Biosciences) for 1 hour at 40° C. Data were collected using a FACS Calibur flow cytometer (BD Biosciences) and analyzed using CellQuest software by gating on the lymphocyte population and calculating the percent of tetramer⁺ cells within the CD8⁺ population. Values represent the tetramer average +/−SEM within each group and were compared to naïve littermate controls (FIG. 4).

### Example 4

### In Vivo Knock Down of Antigen Expression in Naïve Control Mice

As depicted in FIG. 4, immunization with the parent plasmid, pSEM, resulted in a detectable response in mice shown by the presence of 7% Melan-A 26-35-specific CD8⁺ T cells after the plasmid only immunization. The percentage of such cells significantly increased in mice after boosting with the injection of Melan-A peptides, to over 40% of total CD8 cells. In contrast, baseline tetramer positive CD8 cells were detectable in mice immunized with plasmid, pSEM-U6-Melan-A, pre- and post-peptide boosts. This indicates that the expression of Melan-A is inhibited in antigen presenting cells that had taken up pSEM-U6-Melan-A and that such plasmid-driven antigen expression is essential for the induction of immune response in a prime-boost regime. In mice immunized with pSEM-U6-GFP, a reduction of immune response was observed compared to that from pSEM-immunized mice, possibly due to the activation of MAK/interferon a pathway associated with dsRNA. However, significant response (20% tetramer positive CD8 cells) from these mice after peptide boost further verifies the importance of antigen expression from plasmid during the priming event.

### Example 5

### ELISPOT Analysis of In Vivo Knock Down of Antigen Expression in Mice

Instead of measuring cytotoxicity, the CD8⁺ CTL response can be assessed by measuring IFN-γ production by specific effector cells in an ELISPOT assay. In this assay, antigen-presenting cells (APC) are immobilized on the plastic surface of a microtiter well and effector cells are added at various effector:target ratios. The binding of APCs by antigen-specific effector cells triggers the production of cytokines including IFN-γ by the effector cells. The cells can be stained to detect the presence of intracellular IFN-γ and the number of positively staining foci (spots) counted under a microscope.

For ELISPOT assays, all of the immunized animals were sacrificed 7 days after the final injection of peptide. ELISPOT analysis was conducted by measuring the frequency of IFN-γ producing spot forming colonies (SFC). Briefly, spleens were isolated from euthanized animals and the mononuclear cells, after density centrifugation (Lympholyte Mammal, Cedarlane Labs), were resuspended in HL-1 medium. Splenocytes (5×10⁵ or 2.5×10⁵ cells per well) were incubated with 10 μg of Melan-A₂₆₋₃₅ A27L peptide in triplicate wells of a 96 well filter membrane plates (Multi-screen IP membrane 96-well plate, Millipore). Samples were incubated for 42 hours at 37° C. with 5% CO₂ and 100% humidity prior to development. Mouse IFN-γ coating antibody (IFN-γ antibody pair, U-CyTech Biosciences) was used as a coating reagent prior to incubation with splenocytes, followed by the accompanied biotinylated detection antibody. GABA conjugate and proprietary substrates from U-CyTech Biosciences were used for IFN-γ spot development. The CTL response in immunized animals was measured 24 hours after development on the AID International plate reader using ELISpot Reader software version 3.2.3 calibrated for IFN-γ spot analysis.

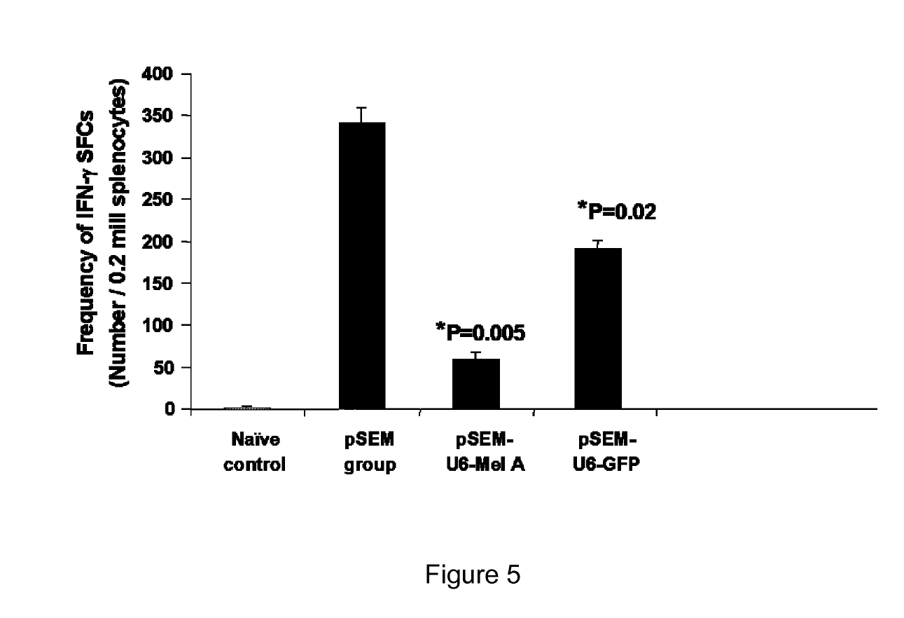

The results as depicted in FIG. 5 show the average IFN-γ spot count for each experimental group. A three fold decrease in spot count was observed in samples from pSEM-U6-Melan-A immunized mice compared to that from mice immunized with pSEM-U6-GFP (p=0.002). This result correlates with that from tetramer assay, suggesting that, lack of antigen expression during plasmid priming significantly abolishes the antigen-specific immune response, quantitatively, as well as qualitatively.

### Example 6

### Control of Autoimmunity Using the Bicistronic Vector

By forming the immunological synapse, the T cell receptor recognizes complexes of MHC with the antigen on the surface of an APC. T cell activation also requires a co-stimulatory signal involving interaction of T cells with B7 family genes on the APC. Furthermore, newly defined signal 3 cytokines (IL12 or IL-1b) can be useful for effector function of T cells.

A bicistronic vector can be used to induce tolerized T cell population and/or T regulatory cells for the control of autoimmunity. By transfecting a pAPC with a bicistronic vector co-expressing an autoantigen and a RNAi that reduces or downregulates a costimulatory signal, (signal 3), or pro-inflammatory molecule, attenuation of T cell activation can be achieved through interference with the immunological synapse, leading to the generation of T-regulatory cells and/or tolerized T cells, and/or T cells in anergy state.

A bicistronic vector is designed and includes a cDNA sequence for an autoantigen that is placed under the control of promoter/enhancer sequence from cytomegalovirus (CMVp), which allows efficient transcription of messenger for the autoantigen upon uptake by cells such as APCs. In addition, the bicistronic vector includes a sequence corresponding to an siRNA for silencing, inhibiting or downregulating the activity of a B7 molecule, which is placed under the control of a U6 promoter.

Administration of the bicistronic vector is used to treat diseases or illnesses such as Type 1 diabetes and multiple sclerosis.

### Example 7

### Promoting CTL Activity by Regulating the T-Regulatory Pathway

A bicistronic vector is designed and includes a nucleic acid sequence that encodes Melan-A₂₆₋₃₅ placed under the control of promoter/enhancer sequence from cytomegalovirus (CMVp). In addition, the bicistronic vector includes a sequence corresponding to an siRNA directed against a B7 molecule, which is placed under the control of a U6 promoter.

The bicistronic vector is administered as a pharmaceutical composition to a population of patients diagnosed with cancer. A second vector that contains a nucleic acid sequence encoding Melan-A₂₆₋₃₅ that does not contain the siRNA for silencing T-regulatory cells is administered as a pharmaceutical composition to a second population of patients diagnosed with cancer. A third vector that does not contain either cistron (Melan-A₂₆₋₃₅ and siRNA against T-regulatory cells) is administered as a pharmaceutical composition to a third population of patients diagnosed with cancer. It is observed that the population to which the bicistronic vector was administered exhibits a CTL response against Melan-A₂₆₋₃₅ that is significantly greater than that observed in the other patient populations.

### Example 8

### Silencing of Immunoproteasomal Activity in Antigen Presenting Cells

A bicistronic vector is designed and includes a sequence for the Melan-A₂₆₋₃₅ A27L peptide antigen placed under the control of promoter/enhancer sequence from cytomegalovirus (CMVp). In addition, the bicistronic vector includes a sequence corresponding to an siRNA for silencing, inhibiting or downregulating the immunoproteasomal activity in antigen-presenting cells (APCs), which is placed under the control of a U6 promoter. The bovine growth hormone polyadenylation signal (BGH polyA) at the 3′ end of the sequence for the Melan-A₂₆₋₃₅ A27L peptide antigen provides a signal for polyadenylation of the messenger to increase its stability as well as for translocation out of nucleus into the cytoplasm for translation. To facilitate plasmid transport into the nucleus after uptake, a nuclear import sequence (NIS) from simian virus 40 (SV40) has been inserted in the plasmid backbone. The plasmid carries two copies of a CpG immunostimulatory motif, one in the NIS sequence and one in the plasmid backbone. Lastly, two prokaryotic genetic elements in the plasmid are responsible for amplification in E. coli, the kanamycin resistance gene (Kan R) and the pMB1 bacterial origin of replication

The bicistronic vector is administered as a pharmaceutical composition to a population of patients diagnosed with cancer. A second vector that contains a nucleic acid sequence encoding Melan-A₂₆₋₃₅ that does not contain the siRNA for silencing immunoproteasomal activity is administered as a pharmaceutical composition to a second population of patients diagnosed with cancer. A third vector that does not contain either cistron (Melan-A₂₆₋₃₅ and siRNA against immunoproteasomal activity) is administered as a pharmaceutical composition to a third population of patients diagnosed with cancer. It is observed that the population to which the bicistronic vector was administered exhibits a CTL response against Melan-A₂₆₋₃₅ that is significantly greater than that observed in the other patient populations.

### Example 9

### Use of a Bicistronic Vector for Gene Therapy Applications

A bicistronic vector is designed and includes a sequence for the Melan-A₂₆₋₃₅ A27L peptide antigen placed under the control of promoter/enhancer sequence from cytomegalovirus (CMVp). In addition, the bicistronic vector includes a sequence corresponding to an siRNA for silencing, inhibiting or downregulating DNA methyltransferase in target cells to which the vector is administered, placed under the control of a U6 promoter. The bovine growth hormone polyadenylation signal (BGH polyA) at the 3′ end of the sequence for the Melan-A₂₆₋₃₅ A27L peptide antigen provides a signal for polyadenylation of the messenger to increase its stability as well as for translocation out of nucleus into the cytoplasm for translation. To facilitate plasmid transport into the nucleus after uptake, a nuclear import sequence (NIS) from simian virus 40 (SV40) has been inserted in the plasmid backbone. The plasmid carries two copies of a CpG immunostimulatory motif, one in the NIS sequence and one in the plasmid backbone. Lastly, two prokaryotic genetic elements in the plasmid are responsible for amplification in E. coli, the kanamycin resistance gene (Kan R) and the pMB1 bacterial origin of replication

The bicistronic vector is administered as a pharmaceutical composition to a population of patients diagnosed with cancer. A second vector that contains a nucleic acid sequence encoding Melan-A₂₆₋₃₅ that does not contain the siRNA for inhibiting DNA methyltransferase activity is administered as a pharmaceutical composition to a second population of patients diagnosed with cancer. A third vector that does not contain either cistron (Melan-A₂₆₋₃₅ and siRNA against DNA methyltransferase activity) is administered as a pharmaceutical composition to a third population of patients diagnosed with cancer. It is observed that the population to which the bicistronic vector was administered exhibits a sustained and persistent CTL response against Melan-A₂₆₋₃₅ that is significantly greater than that observed in the other patient populations.

All references mentioned herein are hereby incorporated by reference in their entirety. Further, embodiments of the present invention can utilize various aspects of the following, which are all incorporated by reference in their entirety: U.S. patent application Ser. Nos. 09/380,534, filed on Sep. 1, 1999, entitled A METHOD OF INDUCING A CTL RESPONSE; Ser. No. 09/776,232, filed on Feb. 2, 2001, entitled METHOD OF INDUCING A CTL RESPONSE; Ser. No. 09/715,835, filed on Nov. 16, 2000, entitled AVOIDANCE OF UNDESIRABLE REPLICATION INTERMEDIATES IN PLASMID PROPOGATION; Ser. No. 09/999,186, filed on Nov. 7, 2001, entitled METHODS OF COMMERCIALIZING AN ANTIGEN; and Provisional U.S. Patent Application No 60/274,063, filed on Mar. 7, 2001, entitled ANTI-NEOVASCULAR VACCINES FOR CANCER.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof

Many variations and alternative elements have been disclosed in embodiments of the present invention. Still further variations and alternate elements will be apparent to one of skill in the art. Among these variations, without limitation, are the specific number of antigens in a screening panel or targeted by a therapeutic product, the type of antigen, the type of cancer, and the particular antigen(s) specified. Various embodiments of the invention can specifically include or exclude any of these variations or elements.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. The skilled artisan can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1-41. (canceled)

42. A vector comprising at least two cistrons, wherein a first cistron comprises a first promoter and a first nucleic acid sequence encoding one or more therapeutic agents, and wherein a second cistron comprises a second promoter and a second nucleic acid sequence encoding one or more RNA molecules that interfere with the expression of a checkpoint protein, wherein the expression of the first sequence is under control of the first promoter and expression of the second sequence is under control of the second promoter.

43. The vector of claim 42 wherein the vector is a plasmid vector or a viral vector.

44. The vector of claim 42, wherein the first promoter is an operably linked promoter/enhancer sequence.

45. The vector of claim 44, wherein the promoter/enhancer is a CMV promoter/enhancer sequence.

46. The vector of claim 42 wherein the one or more RNA molecules that interfere with expression of a biological response modifier is an RNAi, a siRNA, or a shRNA.

47. The vector of claim 42, wherein the second promoter is a U6 promoter sequence.

48. The vector of claim 42, wherein the checkpoint protein is FOXp3, or B7-like molecules.

49. The vector of claim 42 wherein the one or more therapeutic agents comprise an immunogen.

50. The vector of claim 49 wherein the immunogen is selected from the group consisting of tumor associated antigens, tumor specific antigens, differentiation antigens, embryonic antigens, cancer-testis antigens, antigens of oncogenes, mutated tumor-suppressor genes, unique tumor antigens resulting from chromosomal translocations, viral antigens, and fragments thereof.

51. The vector of claim 50 wherein the immunogen comprises a tumor specific antigen or fragment thereof.

52. The vector of claim 50 wherein the immunogen comprises a tumor associated antigen or fragment thereof

53. The vector of claim 52, wherein the tumor associated antigen or fragment thereof is selected from the group consisting of Melan-A, tyrosinase, PRAME, PSMA, NY-ESO-1 and SSX-2.

54. The vector of claim 49, wherein the immunogen consists essentially of Melan-A₂₆₋₃₅, or its analogue ELAGIGILTV.

55. A vector comprising at least two cistrons, wherein a first cistron comprises a first promoter and a first nucleic acid sequence encoding one or more Melan-A epitopes, and wherein a second cistron comprises a second promoter and a second nucleic acid sequence encoding one or more RNA molecules that interfere with the expression of a checkpoint protein, wherein the expression of the first sequence is under control of the first promoter and expression of the second sequence is under control of the second promoter.

56. A method for designing a vector comprising at least two cistrons, comprising placing a first promoter, a first sequence encoding one or more therapeutic agents, a second promoter and a second sequence encoding one or more RNA molecules that interfere with the expression of a checkpoint protein within the same vector, wherein the expression of the first sequence is under control of the first promoter and expression of the second sequence is under control of the second promoter.

57. The method of claim 56, wherein the first and second promoter is selected from the group consisting of a tetracycline responsive promoter, a probasin promoter, a CMV promoter, and an SV40 promoter.

58. The method of claim 56, wherein the vector is a plasmid vector or a viral vector.

59. The method of claim 58, wherein the plasmid is selected from the group consisting of pSEM, pBPL (SEQ ID NO:7) and pROC (SEQ ID NO:8).

60. The method of claim 58, wherein the plasmid is pSEM plasmid.

61. The method of claim 56, further comprising placing an operably linked promoter/enhancer sequence in the vector.

62. The method of claim 61, wherein the promoter/enhancer sequence is a CMV promoter.

63. The method of claim 56, wherein the second sequence is an RNAi hairpin sequence.

64. The method of claim 56, further comprising placing at least one of a reporter gene, a selectable marker, and an agent with immunomodulating or immunostimulating activity in the vector.

65. A mammalian cell transformed with a bicistronic vector of claim 42.

66. A therapeutic composition comprising the bicistronic vector composition according to claim 42.

67. The therapeutic composition of claim 66, further comprising a pharmaceutically acceptable carrier.

68. The vector of claim 42, wherein the one or more RNA molecules that interfere with expression of a biological response modifier comprise an shRNA, and wherein the checkpoint protein is involved in controlling or regulating an immune response.
